SEQUENCE LISTING (3 sequences)

SEQ ID NO: 1 - DNA, 45 bp, primer
  misc_feature (37)..(45): N= A or T or G or C
actctggactaggcaggttcagtggccattatggccnnnnnnnnn

SEQ ID NO: 2 - DNA, 42 bp, primer
  misc_feature (37)..(42): N= A or T or G or C
aagcagtggtgtcaacgcagtgaggccgaggcggccnnnnnn

SEQ ID NO: 3 - DNA, 37 bp, artificial sequence
  linker (1)...(37): linker
ggccgaggcggcctgattaacgatggccataatggcc

(19) United States
(12) Patent Application Publication
(10) Pub. No.: US 2003/0027214 A1
(43) Pub. Date: Feb. 6, 2003

(54) Methods for substrate-ligand interaction screening

(76) Inventor: Carl Alexander Kamb, Salt Lake City, UT (US)

Correspondence Address: MARSHALL, GERSTEIN & BORUN, 6300 SEARS TOWER, 233 SOUTH WACKER, CHICAGO, IL, 60606-6357, US

(21) Appl. No.: 10/162,228

(22) Filed: Jun. 4, 2002

Related U.S. Application Data
(63) Continuation of application No. 09/506,211, filed on Feb. 17, 2000, now abandoned.
Application No. 09/506,211 is a continuation-in-part of application No. 09/251,364, filed on Feb. 17, 1999.
Application No. 09/506,211 is a continuation-in-part of application No. 09/350,419, filed on Jul. 8, 1999.

(51) Int. Cl. G01N 33/53; C07K 14/00; C07K 7/08; G01N 33/543

(52) U.S. Cl. 435/7.1; 436/518; 530/324; 530/333

(57) ABSTRACT

Provided by the present invention are novel methods of detecting substrate-ligand interactions, and more specifically relates to methods for detecting and characterizing polypeptide-ligand interactions. By practice of this invention, protein interaction maps may be generated for humans or for other organisms.

### RELATED APPLICATIONS

This application is a continuation in part of U.S. application Ser. Nos. 09/251,364 and 09/350419 of K. A. Kamb, entitled “Methods For Substrate-Ligand Interaction Screening,” and claims priority therefrom. The disclosures of the priority applications are incorporated by reference in their entirety herein.

### FIELD OF THE INVENTION

The present invention relates generally to novel methods of screening for, detecting, identifying and quantifying substrate-ligand interactions, and more specifically relates to novel methods for achieving these ends for protein-ligand interactions, and more specifically protein-protein interactions. The inventive method is suitable for screening large or very large libraries, and for generating protein interaction maps.

### BACKGROUND OF THE INVENTION

Many physiological functions in mammals and other organisms are mediated through interactions of cellular proteins with a variety of endogenous ligands, including for example other proteins, glycoproteins, polypeptides, hormones or other small molecules. Because of the importance of these endogenous protein-ligand interactions, pharmaceutical companies often seek to modify or disrupt physiological pathways by providing exogenous molecules that interact with those endogenous proteins. In some cases, researchers may target particular, previously characterized proteins, and screen for molecules that interact with that protein. But in the vast majority of cases, researchers lack the initial insight into a given physiological pathway, and must first identify the native proteins involved in that pathway before achieving the ability to modify the physiological effects of that pathway.

While much is now known about the genome of humans and other organisms, researchers have yet to close the link in many instances between DNA sequence information and physiological function. In order to do so efficiently, it is desirable to first identify key native proteins that are related to specific physiological functions, and then to relate those proteins to the DNA sequences encoding them. Once such key proteins are identified, then researchers may identify ligands (proteinaceous or otherwise) that interact with these proteins, and in turn relate these targeted protein-ligand interactions to physiological changes. But to date, the methods used in the art for evaluating protein-ligand interactions have not provided a simple, efficient method of identifying the key native proteins (and screening for ligands that interact with them). Nor has the art provided an efficient high-throughput screening method that allows researchers to broadly catalogue, e.g., all endogenous protein-protein interactions, before turning to the related questions of physiological function and targeted drug development.

Researchers are particularly hampered in their ability to comprehensively catalogue endogenous protein-protein interactions in a human or other organism by the sheer magnitude of endogenous proteins that must be evaluated. For example, some 10⁵-10⁶ proteins are believed to be encoded by the human genome. To begin by evaluating the interaction of each of those proteins with each other encoded protein thus requires evaluating 10⁶×10⁶ protein-protein interactions, or 10¹² total interactions. Such a large-scale evaluation is problematic, because it involves evaluating a matrix of all possible combinations; thus the number of interactions scales as the square of the number of proteins to be evaluated (termed more generally herein, the “n×n” problem). Current methodologies simply cannot evaluate such vast numbers of protein interactions in a time- and cost-efficient manner. The inability of current methodologies to provide rapid, quantitative high-throughput screening is particularly acute if comparative information regarding protein interactions in different cell types or cell states is desired.

The limitations of current methodologies can be seen by considering current technologies for mapping protein interactions. For example, one typical approach to probing protein-ligand interactions involves an in vivo, quasi-genetic approach known as the yeast two-hybrid assay. This approach suffers from the drawbacks of (i) limitation to probing protein-protein interactions, (ii) lack of speed, (iii) prevalence of false-positive and false-negative results, (iv) lack of quantitative information (e.g., binding affinities between specific protein pairs). These drawbacks remain a substantial obstacle to utilizing yeast two-hybrid technology to screen for interactions, notwithstanding recent advances in, e.g., automation of the two-hybrid technology.

Phage display techniques have been used to select proteins that bind to a particular, pre-selected ligand. Such methodologies again are essentially in vivo, as the proteins that are borne by the phages are isolated and identified only after the intermediate steps of culturing the phage in E. coli, plating the bacteria and isolating phage from phage-generated plaques or cultures. These intermediate steps are necessary because the phage must be generated in cells and cannot be created without cells. In addition, phage must be bound, eluted, and re-grown in cells prior to analysis. Thus, the technique is not well suited to screening applications such as generating protein interaction maps. Nor is the technique amenable to high throughput applications. Moreover, the technique does not provide quantitative information.

Alternatively, researchers have utilized limited-throughput screening techniques to evaluate the binding of ligands to a particular substrate. For example, a selected proteinaceous substrate, or small number of such substrates, have been immobilized by a variety of means for exposure to a select pool of ligands. E.g., U.S. Pat. Nos. 5,635,182; 5,776,696; 5,498,530; Major, E. S., “Challenges of high throughput screening against cell surface receptors,” J. Recept. Signal Transduct. Res. 15(1-4):595-607 (1995). But such methodologies are not amenable to screening, e.g., large or very large populations, for generating protein interaction maps, and/or for screening previously uncharacterized substrates—i.e., the techniques do not adequately address the “n×n” problem generated by large-scale screening efforts.

More generally, other researchers have utilized various solid-state screening techniques to evaluate interactions of different moieties. For example, assays exist that immobilize known antigens or antibodies on beads or other such solid supports. E.g., Roque et al., Acta Histochem. 98(4):441-451 (Nov. 1996). Two or three-dimensional matrices tagged with nucleic acids have been utilized to screen for DNA-binding moieties. Other researchers have utilized “lawn assays” that detect protein interactions utilizing diffusion of a ligand through a colloidal matrix. However, none of these techniques addresses the “n×n” problem, and thus none provides rapid, quantitative and/or large-scale evaluation of substrate-ligand interaction, or more specifically, protein-protein interactions.

Thus, the need remains for a flexible, efficient, quantitative methodology for evaluating substrate-ligand interactions generally, and protein-protein interactions in particular. The present invention meets such needs.

### SUMMARY OF THE INVENTION

The present invention provides methods for detecting substrate-ligand interactions, more particularly polypeptide-ligand interactions or polypeptide-polypeptide interactions. The polypeptides may be individual polypeptides, or may alternatively be library polypeptides, including those of large or very large libraries and/or of native, endogenous polypeptides. The methods utilize randomizable ligand-bearing supports bearing unique tags, and may optionally use location-determinable supports. In some embodiments, a magnetic support may be used to adhere to either the substrate or the ligand, and magnetic culling of bead aggregates that result from substrate-ligand complexes provides for an enrichment step. Interacting pairs are identified by correlating (i) location information and (ii) identity information provided by each unique tag. The location information may be derived from correlating back to a unique location, or alternatively by evaluating the origination of location-determinable supports. The unique tags may use a variety of techniques, including fluorescent bar codes, to encode ligand identity information. By such methods, protein interaction maps for, e.g., the human organism, may be generated.

The invention further provides methods for identifying and quantifying such interactions. In some embodiments, the interacting substrate-ligand pairs may be detected with antibodies, for example fluorescent antibodies, and the interactions quantified via a FACS machine or CCD camera.

### BRIEF DESCRIPTION OF THE DRAWINGS

FIG. 1 is a map of plasmid vector pSE420/trx/GFP.

FIG. 2 is a map of plasmid vector pSE420/biotrx/GFP/BirA.

FIG. 3 is a map of plasmid vector pSE420/Caltrx/GFP.

FIG. 4 is a map of plasmid vector pSE420/DHFR/GFP.

FIG. 5 is a map of plasmid vector pLex biotrx GFP LbirA.

FIG. 6 depicts a bead that has been derivatized for crosslinking with a methotrexate as an adhesion moiety and SANPAH as a photoactivatable crosslinker.

FIG. 7 is a FACS histogram demonstrating the crosslinking of interacting proteins. Peak A is streptavidin coated particles reacted with BL21 lysate and FITC-calmodulin conjugate. Peak B is streptavidin coated particles reacted with a lysate having a biotin-thioredoxin-CBP fusion protein, which is then exposed to the FITC-calmodulin conjugate in the presence of calcium chelator EGTA. Peak C is streptavidin coated particles reacted with a lysate having a biotin-thioredoxin-CBP fusion protein, which is then exposed to a FITC-calmodulin conjugate. Peak D is streptavidin coated particles reacted with a lysate having a biotin-thioredoxin-CBP fusion protein, FITC-calmodulin conjugate and a protein crosslinking agent. Peak E is streptavidin coated particles reacted with a lysate having a biotin-thioredoxin-CBP fusion protein, FITC-calmodulin conjugate, protein crosslinking agent and then EGTA.

FIG. 8 depicts the enrichment of biotin-coated fluorescent beads from a mixture of fluorescent beads coated only with Bovine Serum Albumin (BSA), using streptavidin-coated magnetic beads. The streptavidin and the biotin interact, and subsequently the aggregates are segregated from the BSA-coated beads with a magnet.

FIG. 9 depicts the enrichment of beads coated with an SV40 large T antigen conjugate from a mixture of fluorescent beads coated only with BSA, using magnetic beads coated with an anti-SV40 large T antigen antibody conjugate. The antigen and antibody interact, and subsequently the aggregates are segregated from the BSA-coated beads with a magnet.

### DETAILED DESCRIPTION OF THE INVENTION

The methodologies of this invention provide rapid, efficient, quantitative substrate-ligand interaction screens. The invention differs from prior approaches in that it does not rely on yeast-two hybrid technology or other such in vivo techniques, but instead provides a high throughput in vitro screening methodology. While the inventive methods do provide rapid, quantitative screening of individual polypeptides or other substrates against a selected ligand pool, the techniques provide for scale-up for screening small (on the order of 1×10²) substrate populations, and advantageously may be used to screen large (on the order of 10³ or 10⁴) or even very large (10⁵, 10⁶ or even 10⁷) populations. This is so because the inventive use of both location information and unique tags to identify substrate-ligand pairs renders the technique suitable for screening previously uncharacterized polypeptides or other substrates en masse, rather than relying upon the pre-selection of a known substrate or small number of substrates and thereby screening in a “1×n” manner rather than an “n×n” manner.

More specifically, the invention provides its quantitative, high throughput polypeptide/ligand screening capabilities by cross-indexing (i) polypeptide (or other substrate) identity information derived from the characteristic, unique location from which one particular polypeptide (or other substrate) is derived, and (ii) ligand identity information derived from its associated randomizable support, which bears a unique tag that correlates to the identity of that ligand. The polypeptide may be an individual polypeptide, or alternatively may be a member of a polypeptide library of various sizes. Non-polypeptide substrates may include, e.g., small organic or inorganic molecules, of either endogenous or synthetic origin.

In some embodiments, a unique polypeptide or other such substrate may be adhered to a location-determinable support, which correlates to the unique location from which a particular library polypeptide is derived, prior to exposure to the ligands. In other embodiments the unique polypeptide or substrate remains in a lysate or other such solution, to which the randomizable ligand-bearing supports are added. The supports described herein may be microbeads, or may be a fixed solid support. The unique tag that identifies a particular ligand may be, for example, a fluorescent “bar code” or oligonucleotide tag.

The invention encompasses a number of potential substrates, including (i) non-nucleic acid, proteinaceous substrates such as individual polypeptides and library polypeptides, (ii) other non-nucleic acid substrates such as exogenous natural products, exogenous small organic molecules or endogenous non-proteinaceous products, (iii) nucleic acid substrates, and (iv) inorganic substrates. The term “individual polypeptide” refers to an amino acid sequence, for example a protein or protein domain, and also includes further derivatized amino acid sequences, such as, e.g., glycoproteins. The sequence may be that of a native molecule (i.e., endogenous to a given cell), or alternatively may be synthetic. Individual polypeptides are typically identified and characterized in advance of the ligand screening, and are not generated or screened en masse. Library polypeptides encompass the same sorts of amino acid sequences, but are encoded by DNA sequences that are generated and screened en masse, and may be previously unknown or uncharacterized molecules. The libraries may vary in size, and include large or very large libraries. In particular, the library polypeptides may include all or substantially all native protein domains encoded by the human genome, or expressed in the human organism. As termed herein, “ligands” are molecules that are screened to identify those members that interact with the polypeptides or other substrates. Ligands may be proteinaceous moieties such as, e.g., polypeptides or glycoproteins from a variety of sources, or may be other organic or inorganic molecules. The ligands may be endogenous molecules such as hormones, antibodies, receptors, peptides, enzymes, growth factors or cellular adhesion molecules, or may be derivatized or wholly synthetic molecules. Because of the flexibility of the invention, the identity of the ligands need not be known or preselected in advance, and may also be large or very large populations.

The present invention lends itself to automated high-throughput embodiments, in which microbeads serve as the location-determinable and/or randomizable supports. Such microbeads may be readily dispersed by robotic means to, e.g., 384-well microtiter plates. The polypeptides or other such substrates interact with ligands to form interacting pairs, termed “complexes” herein. When each member of the interacting pair are immobilized on supports, then the two supports are linked via the substrate/ligand complex to form an “aggregate.” The aggregates and/or complexes are then sorted and identified. Means for accomplishing this include a CCD camera or a fluorescence-activated cell sorter (FACS).

The speed and selectivity of this inventive methodology may be further enhanced by utilizing magnetic attraction to facilitate a solid-state interaction between the polypeptide or other substrate that is bound to a location-determinable support, and the ligand that is bound to the randomizable support. This may be accomplished by utilizing a magnetic material for the support, and then collecting the complexes or aggregates by culling the magnetic supports with a magnetic force, for example by applying a magnetic field to the exterior of the arrays or by inserting a magnetized body such as a pin into each well of the array.

Because the methodologies of the present invention are so rapid and efficient, screening is not limited to small, pre-characterized or artificially culled substrate populations, nor does the invention require pre-selection of known ligands of interest. Rather, the invention allows for high throughput cross-screening of large or very large populations—e.g., the entire endogenous protein library of a human organism. Indeed, the methodologies of this invention are particularly well-suited for large-scale screening of some 1×10⁶ proteins, which is the estimated number of proteins produced in a human being. Thus, the inventive methods and materials answer a long-felt need in the industry for evaluating the interactions of endogenous proteins within a human organism, to form a comprehensive human “protein interaction map.” Alternatively, the inventive methodology may be used to screen the selected library polypeptides against other ligand libraries—for example, endogenous ligand libraries such as a second polypeptide library, endogenous hormones, antibodies, receptors, peptides, enzymes, growth factors or cellular adhesion molecules, or on the other hand exogenous ligands derivatized or wholly synthetic molecules, natural products, synthetic peptides, or synthetic organic or inorganic molecules.

Other uses and advantages of this screening methodology will be apparent to those of skill in the art.

Overview of the Methodology

The general strategy of the methodology is exemplified as follows. A substrate pool of interest is selected—for example, a library of all or substantially all native polypeptides expressed by the human organism, or a selection of individual polypeptides of interest. A corresponding set of library polypeptides or individual polypeptides are generated in cells. Single colonies, each of which is expressing one particular polypeptide of interest, are selected and replated in order to generate single-cell clones (i.e., multiple copies of one particular cell, each cell expressing the same individual polypeptide or unique member of the polypeptide library). Each such clone is uniquely located at one particular location of an array—e.g., each particular well of a given 384 well plate contains a one particular clone. The expression products of each of those clones are then harvested from the cells, for example by generating soluble lysates that correspond to each of the plated clones. Thus, each well corresponds to the soluble lysate of one particular clone, which in turn corresponds to one individual polypeptide or one unique member of a polypeptide library. Alternatively, each member of a non-proteinaceous substrate pool of interest is individually arrayed at a unique location.

In the case of proteinaceous substrates, the expression product of each lysate is then either (i) kept segregated in a unique location (e.g., one particular well of a 384 well array); or (ii) exposed to a solid support that is unique to that lysate source, and whose location may be tracked in order to identify the corresponding lysate source to which it was exposed. Such a solid support is termed herein, a “location-determinable support.” This location-determinable support may be any solid support that is suitable for adhering a desired polypeptide from a polypeptide-containing lysate, and which can be correlated back to a particular polypeptide source—e.g., a particular microtiter well in a particular array. Exemplary location-determinable supports include (i) beads that are kept segregated in microtiter wells that are derived from, and thus correspond to, the original lysate-bearing array location; and (ii) a fixed solid support such as a pin or other such probe that is suitable for dipping into one unique location in a lysate-bearing microtiter well. The same strategy may be applied to non-proteinaceous substrates.

The ligands to be screened may advantageously may be immobilized on a solid support, although in order to screen a large variety of ligands for interaction with any particular substrate, such solid supports should be “randomizable”—i.e., in terms of this invention, (i) each such support can be dispersed into a mixture of such supports in a manner that allows for full mixing and resultant random distribution of support constructs in any subsequent aliquot of the mixture, and (ii) each such randomizable support bears with it a corresponding unique identification tag that identifies the associated ligand. Use of such randomizable supports to create a fully integrated set of ligand-bearing supports increases the statistical likelihood that an aliquot taken from the fully integrated ligand set will contain a fully dispersed, representative subset of ligands. Examples of such randomizable supports include microparticles (e.g., small beads) in a variety of materials and sizes. The unique tags may be, for example, fluorescent, oligonucleotide sequence tags, mass tags, radio tags, or any combination thereof.

As one exemplary use of the invention, a polypeptide library may be screened against itself to generate a “protein interaction map”—i.e., an “n×n” matrix of interactions for all or substantially all native polypeptides of a human or other selected organism. By “native polypeptides” is meant polypeptides that are endogenous to a selected organism—i.e., that are encoded by the organism's genome and which may be expressed by that organism. Native polypeptides include functional subunits or “protein domains” of endogenous proteins. In such embodiments, the polypeptides of interest serve as both substrate and ligand—i.e., each randomizable support is adhered to multiple copies of one member of the polypeptide library, and each unique array location contains multiple copies of one member of the polypeptide library. Once each randomizable support bears its corresponding unique library polypeptide, the supports are pooled into one volume and mixed to form a fully integrated ligand collection—i.e., the pooled volume represents all ligand species. Next, ligand aliquots are drawn from this fully integrated ligand collection. Each aliquot contains a randomized, representative sampling of the ligands that is statistically likely to contain at least one copy of each species of ligand present in the pooled ligand volume. These ligand aliquots then are presented for interaction with each of the library polypeptides, either by simply adding an aliquot of integrated ligand-bearing supports to each uniquely located library polypeptide lysate within the library array, or by first adhering the library polypeptides in the array to location-determinable supports and then exposing each such set of polypeptide-bearing supports (which bear only one type of polypeptide) to an integrated aliquot of randomizable supports.

In another exemplary use a first set of library polypeptides may be screened against a second, independent polypeptide library, composed of, e.g., a separate set of native protein domains, a set of synthetic polypeptides containing, e.g., point mutations, or randomly generated synthetic polypeptide sequences. In such embodiments, the same methodology is applied, but a second, independent expression library is used to generate a second, independent array containing the second, independent polypeptide library.

In another exemplary use, a first set of polypeptides may be screened against some other ligand set—e.g., small organic molecules, natural products, hormones, receptors, antibodies, peptides, enzymes, growth factors, cellular adhesion molecules, combinatorial library components and the like—that is adhered to the randomizable support and presented to the library polypeptides. In many such instances, a prior cellular expression step to produce the ligands will not be necessary.

Whatever the source of the ligands that are adhered to the randomizable supports, the methodology is completed by exposing each uniquely located substrate (either in solution or adhered to its analogous location-determinable support) to an aliquot of ligand-bearing supports. If the ligand bearing support is exposed directly to a substrate, e.g., to a lysate or other such polypeptide-bearing solution, then any interactions will result in formation of a substrate-ligand complex—e.g., a randomizable support with consecutive layers of adhered ligand and polypeptide. If the substrate is first immobilized on its own support, then any substrate-ligand interaction will adhere the two supports into an aggregate. Such aggregates may be detected and characterized in that form. Alternatively, the aggregates may be resuspended in a corresponding unique library polypeptide solution to displace the support-linked polypeptide with an unbound form of that polypeptide, or removed by some other procedure.

Interactions between substrates and ligands are then detected by fluorescent or other means, for example by use of a fluorescently tagged antibody. Interacting pairs are then culled out in a sorting or detection process, for example via FACS, so that the components of the various complexes may be identified. The identity of the substrate is determined by correlating it to the unique array location from which it was derived (either directly, or via the analogous location-determinable support). If the substrate is proteinaceous, then the DNA encoding the polypeptide produced by the original single-cell clone at that unique location of the library array may then be sequenced or otherwise characterized. The identity of the ligand is determined by evaluating the associated unique identification tag on the randomizable support to which that ligand is bound. If the ligands are also polypeptides that have been uniquely arrayed, the unique identification tag can be further correlated back to a single clone in its corresponding array location.

The screening methods of the present invention can be adapted in a number of ways apparent to those of skill in the art to displacement screening. In one non-limiting embodiment, the substrate-ligand pairs are first formed, and are adhered to a solid support. Subsequently, these pairs are exposed to a secondary ligand. If the secondary ligand is capable of adhering to the substrate, then in many cases it will displace the first ligand. The substrate-secondary ligand pair can then be manipulate, enriched and analyzed according to the method of the invention. The secondary ligand may be a proteinaceous moiety such as, e.g., a polypeptide or glycoprotein from a variety of sources, or may be some other organic or inorganic molecule. The secondary ligand also may be an endogenous molecule such as a hormone, antibody, receptor, peptide, enzyme, growth factor or cellular adhesion molecule, or may be a derivatized or wholly synthetic molecule. In particularly preferred embodiments of displacement screening, the secondary ligand is a small organic molecule.

Generation and Expression of Polypeptide Fusion Libraries

If the substrate of interest is proteinaceous, then an expression library may be generated first. The overall goal of this step is to generate a selection of desired individual polypeptides or library polypeptides that are suitable as either substrate or ligand (or both), for rapid, efficient ligand interaction screening. Once a desired pool of polypeptides is identified, DNA encoding each member polypeptide is incorporated into a corresponding expression construct that produces the desired levels of protein expression. If it is desired to adhere the polypeptides to a support (e.g., to a bead acting as either a location-determinable support or as a randomizable support), then the DNA encoding each member polypeptide is fused in frame with DNA encoding a suitable adhesion partner to form a polypeptide/adhesion moiety fusion construct, described elsewhere herein. Optionally, as described in more detail below, the construct may also utilize a downstream marker that provides rapid indication of whether the fusion construct is in fact expressed in frame, and with no premature terminations, and/or in a stable, suitably folded conformation.

In the case of screening the native cellular proteins of an organism, an expression library is created by standard techniques, generating a sufficient number of fragments of DNA so as to ensure that all protein domains are likely to be expressed in the library. Sambrook, Molecular Cloning: A Laboratory Manual, 2nd ed., Cold Spring Harbor Laboratory Press (1989), Chapters 7-9. Genomic DNA, cDNA synthetic or cloned DNA sequences may be used. As one non-limiting example, synthesis of cDNA and cloning are accomplished by preparing double-stranded DNA from random primed mRNA isolated from, e.g., human placental tissue. Alternatively, randomly sheared genomic DNA fragments may be utilized. In either case, the fragments are treated with enzymes to repair the ends and are ligated into an expression vector suitable for expression in, e.g., E. coli cells. Exemplary vectors include inducible systems, e.g., the trc promoter system, which is induced by addition of suitable amounts of IPTG.

If a subcloning strategy is to be employed, the library polypeptide-encoding vectors may be introduced into E. coli and clones are selected. Before proceeding with the inventive method, the quality of the selected library optionally may be examined. For example, a set of 100 clones can be picked and sequenced at random, looking for homologies to known genes, evidence of splicing, and such features. Alternatively, the library representation can be explored by filter hybridization using probes of sequences of known abundance such as actin and tubulin. These sequences should be present at a frequency in the library of between 0.01% and 1.0%.

Once a satisfactory polypeptide-encoding library (or, alternatively, DNA encoding a desired set of individual polypeptides) is obtained, DNA encoding a suitable adhesion moiety may be incorporated in frame with the polypeptide encoding DNA sequences. This DNA fusion construct is then placed under control of a selected promoter in an expression vector construct, so that upon induction one obtains suitably high levels of expression of the fusion construct. There are many suitable adhesion moieties known to the art, including without limitation biotin/avidin, thioredoxin/PAO, calmodulin binding peptide/calmodulin, dihydrofolate reductase/methotrexate, maltose-binding protein/amylose, chitin-binding domain/chitin, cellulose-binding domain/cellulose, glutathione-S-transferase/glutathione, or antibody/antibody epitopes such as the FLAG epitope. One of ordinary skill may choose an adhesion moiety that binds either reversibly or irreversibly to its complementary moiety. One factor to consider in selecting an adhesion moiety complex is the relative spontaneous dissociation constants (K_{D}) of the complexes. For example, the biotin/avidin link has a K_{D} of approximately 10⁻¹⁵ M and is therefore relatively stable and irreversible. Maltose binding protein/amylase, on the other hand, is less stable, with a K_{D} of 10⁻⁶ M One option to increase stability is to use cross-linking, for example by selecting a fusion protein with an adhesion moiety that can be cross-linked by UV light.

The expression vector is chosen based largely on its ability to generate moderate to high expression levels of either a given polypeptide or a fused polypeptide/adhesion moiety (termed herein, a “fusion construct”), in a host cell of interest. E. coli is one such host cell, although those of skill will appreciate that other bacterial, yeast or mammalian host cells, for example 293 cells, are also suitable for use in the present invention. In the case of E. coli, many suitable expression vectors are known to those in the art. For example, the expression vector may employ the P_{L}, P_{R}, P_{lac}, P_{tac}, P_{trc}, Pₜᵣₓ or T7 promoters, to name only a few such promoters known to those in the art. These promoters are regulated such that high level expression is induced via increased growth temperature (from P_{L} or P_{R} through a mutant temperature-sensitive form of the lambda repressor, cI857) or by addition of a suitable inducing agent (e.g., IPTG for P_{lac} or P_{tac}) to the media. In order to provide a recognition sequence for detecting interacting polypeptide/ligand pairs, the expression vector may optionally be constructed to produce a fusion protein that consists of an N- or C-terminal recognition domain (for example, an epitope that is specifically recognized by an antibody), followed in frame by a sequence encoding the desired library polypeptide which is optionally flanked by sites to facilitate cloning, followed by an N- or C-terminal adhesion domain to enable attachment to a solid support, depending on the strategy employed.

Optionally, the expression vector may include a suitable downstream marker such as a reporter or antibiotic resistance gene, by which one may determine whether the expression vector construct is intact and correctly in frame. This variant includes in the above-described DNA fusion construct an additional marker sequence designed to sort out viable constructs from, e.g., out of frame or inverted constructs. Suitable reporter sequences include green fluorescent protein, which is one of a family of naturally occurring fluorescent proteins whose fluorescence is primarily in the green region of the spectrum, or modified or mutant forms having altered spectral properties (e.g., Cormack, B. P., Valdivia R. H. and Falkow, S., Gene 173: 33-38 (1996)). (Both native GFP and such related molecules are collectively referred to herein as “GFP”) Alternatively, this GFP reporter may be inserted into the expression construct in place of the adhesion domain if only the integrity of the library polypeptide-encoding portion of the construct is of interest. Non-fluorescent markers of construct integrity may also be employed, including a variety of antibiotic resistance genes that are familiar to the art.

Fluorescent reporters such as GFP allow for subsequent rapid sorting of expression products using flow cytometry with a fluorescence-activated cell sorter (FACS) machine. This FACS sorting detects expression constructs that properly read through the GFP reporter sequence and which are expressed at desirably high levels. Cells that express intact, in-frame constructs are readily separated by detecting and collecting “bright” cells, which have an intact GFP moiety that is properly in-frame with the polypeptide of interest, correctly folded, and located downstream from a functional promoter. Constructs that are not intact will be dim. Similarly, constructs with mutations or frame-shift deletions will eradicate the proper relationship of the GFP moiety to the promoter, and the cells bearing such constructs will be dim. Collecting only bright cells in this enrichment step significantly reduces the number of underexpressed or nonfunctional fusion polypeptides that proceed into subsequent screening steps. If antibiotic resistance is used as a marker, then transformed cells are plated on antibiotic-bearing media; only those cells that read through completion of a construct that includes an intact, downstream antibiotic resistance gene will survive and grow.

After the GFP-expressing clones are isolated, the polypeptide or fusion construct inserts can be recovered. If the polypeptide library/adhesion moiety DNA fusion construct was screened, the GFP reporter sequence may optionally be deleted from the vector using standard restriction endonuclease fragment excision and religation, or other such techniques. If only the library polypeptide-encoding constructs were screened but fusion to an adhesion moiety is desired, then the polypeptide-encoding fragments are transferred into a vector containing the adhesion-domain, or alternatively, the adhesion-domain-encoding sequence can be inserted into the vector, or swapped into the vector in exchange for the GFP reporter sequence. Other markers such as antibiotic resistance genes may similarly be removed, if desired.

Generation of Individual Arrays

Next, each substrate must be individually arrayed at a unique location. In the case of proteinaceous substrates, each corresponding clone is arrayed separately, in a unique location, so that in subsequent steps, the identity of any particular polypeptide may be determined by cross-referencing back to its unique location in the original array. Non-proteinaceous substrates may be arrayed directly, without a preceding expression step.

In order to obtain a source of only a given single polypeptide, a single-cell clone is obtained as follows. Once the above-described DNA fusion constructs are assembled, selected host cells are transfected or transformed by standard gene transfer techniques such as electroporation. The transformed cells are selected by growth of colonies on selective media familiar to those of skill in the art (e.g., standard ampicillin-enriched Luria Broth). Single colonies are then picked and placed into growth media in, e.g., 384-well microtiter trays. A robot may be used for this purpose. If desired, duplicate trays may be prepared bearing host cells of identical clones in identical array locations on a separate set of microtiter trays. (Duplicate arrays are particularly desired if the ligands to be screened also are polypeptides—i.e., if a protein-interaction map is sought).

As a result of this step, library arrays in, e.g., 384-well plate format are generated in which each well produces a unique polypeptide (derived either from a library, or from a selection of individual polypeptides of interest). Thus, in later steps, the identity of a particular polypeptide may be determined by tracing its origin back to this corresponding unique array location.

Generation of Lysate Plates

Once each desired polypeptide is being expressed by the corresponding host cells, the cells are lysed so as to release the polypeptides. This growth and lysis may be accomplished directly, in each unique array location that contains them (e.g., microtiter well). Alternatively, in some embodiments each single-cell clone may be grown in an intermediate location of larger size or volume, so that a greater number of cells may be generated and concentrated for lysis. In such embodiments, each concentrated volume of polypeptide is then either lysed and the lysate transferred to its corresponding, unique array location, or the concentrate is transferred to that array location and then lysed in situ. Each clonal lysate then is kept separate from every other, and in a unique location that can be referenced throughout the ligand screening process. Thus, each soluble lysate can be correlated back to its unique library array location, and the identity of the library polypeptide ascertained thereby, as the soluble lysates are used in later ligand interaction screening steps.

In order to obtain the uniquely arrayed soluble lysates, the host cells first are grown until mid- or late-log phase. Expression of the DNA fusion constructs (library polypeptide and adhesion moiety) is induced by whatever method is required by the selected promoter (e.g., IPTG or by raising the growth temperature to 42° C.). After one to five hours of continued cell growth under inducing conditions, the cells are lysed to free the library polypeptide/adhesion moiety fusion constructs.

Any methods familiar to those of the art may be used to free the polypeptides of interest from the host cells. For example, the host cells may be treated with lysozyme to remove the cell wall, followed by hypotonic shock to disrupt the cell membranes and release the contents of the cell into the buffer. The cells alternatively may be sonicated, lysed with a freeze/thaw protocol, or lysed by addition of detergent. The lysate may optionally be concentrated by standard techniques prior to further process steps. Alternatively, the library polypeptide or its corresponding fusion construct may be secreted by the cells, in which case the growth media rather than the cells are further processed.

Other Ligands

In some embodiments of the invention, the screening may seek to identify interacting pairs of endogenous polypeptides, in which case duplicate sets of soluble lysate arrays may be generated from the same set of library polypeptides. In other embodiments, a variety of other ligands may be tested for interaction with the original library polypeptides or other substrates of interest. These other ligands may be proteinaceous in nature, in which case the above procedure may be modified slightly so that a set of host cells expressing the proteinaceous ligands is generated, and the corresponding array obtained.

In other cases, exogenous ligands may be screened for interaction with the polypeptides of interest. Ligands such as small molecules, natural products, hormones, receptors, antibodies, peptides, enzymes, growth factors, cellular adhesion molecules, combinatorial library components and the like may be exposed directly to an appropriate randomizable support (e.g., a support that will adsorb sufficient amounts of the ligand). In other instances, the ligands may require initial derivitization so as to be chemically reactive with surface functional groups on the support, in which case the ligands are, e.g., covalently linked to the support. Alternatively, the ligands may be synthesized on the support. Alternatively, this screening methodology can be altered slightly to serve as a displacement assay, wherein a secondary ligand such as a small molecule is exposed to the primary ligand/substrate pair. The secondary ligand may advantageously be adhered to a randomizable support with a unique tag (for embodiments in which a large or very large number of such secvondary ligands are screened). Alternatively, for embodiments in which a lesser number of secondary ligands are screned, such secondary ligands can be free in solution. In either event, pairs in which the secondary ligand displaces the primary ligand can be detected, collected and analyzed as described elsewhere herein.

Preparation of Randomizable Supports with Unique Tags

In order to screen a variety of ligands for interaction with a given polypeptide, the method generally requires using a support or substrate that will serve three functions; (a) it will adhere to the ligand of interest; (b) it will be fully randomizable, so that an aliquot containing a representative sampling of ligands may be presented to each polypeptide of interest, and (c) it will carry a unique identification tag that corresponds to the particular ligand adhered to its surface, and distinguishes it from other ligand-bearing supports.

In one embodiment of the invention, the randomizable support is a bead or other such microparticle. A variety of bead sizes and compositions are suitable for use in the present invention. For example, bead size may range from 50 nm to 50 microns in diameter. The beads may be composed of polystyrene, glass (silica), latex, agarose, magnetic resin, or a variety of other matrices. Some beads may be obtained from commercial sources with adhesion moieties already attached; for example, numerous avidin-conjugated beads are available. Other beads can be obtained with functional groups such as hydroxyl or amino groups suitable for chemical modifications, such as attachment of adhesion moieties that will interact with the fusion protein. In yet another formulation, the beads do not require specific functional groups; rather, the interaction between the fusion protein and the bead is of a nonspecific type involving, e.g., hydrophobic interactions. Beads suitable for this purpose may be polystyrene, latex, or some other plastic.

If the beads require functionalization in order to bind to the selected polypeptide or ligand, then enough beads are generated in one reaction to permit numerous experiments to be performed, e.g., 10¹⁴ beads. These beads are then stored under conditions that ensure the stability of the chemical modifications, such as low temperature. For example, in mapping protein interactions in a human cell, approximately 1×10⁷ beads are generated for each potential expression product to be screened (e.g., in the case of the human cell, approximately 1×10⁶ potential endogenous polypeptides, resulting in a need for some 1×10¹³ beads). This number of beads ensures that at least one full experiment involving genome-wide protein-protein interaction measurements can be performed.

A variety of methods are suitable for providing each support with an identification tag that correlates to the ligand that the support will bear. For example, the beads may be tagged with DNA tags in which the tags can be amplified and fingerprinted, or detected by hybridization. Alternatively or in conjunction, the beads may be tagged with fluorescent tags such as fluorescent barcodes, radio frequency tags, or mass tags detected by mass spectrometry

Fluorescent Barcodes

Fluorescent tags for the randomizable supports are advantageous because the identification tag may be read simultaneously with quantification of the binding interaction. One representative method of fluorescent tagging is to use the variety of existing fluorescent materials such as fluorescent organic dyes or microparticle dyes, and the sensitivity of existing fluorescence detectors, to devise a series of fluorescent barcodes.

Fluorescent barcodes may be generated as follows. Fluorescence detectors presently exist that can quantify fluorescence at up to nine separate wavelengths using multiple lasers, photo-multiplier tubes (PMTs) and filter sets. One example of such a device is the Cytomation flow cytometer that is not only capable of measuring fluorescence at multiple wavelengths in single cells or beads, but also of sorting cells and beads based on these signals. The measurements are also highly accurate, so that it is possible to distinguish easily a fluorescence value of 0 (background) from, 1×, 2×, 3×, and 4×. Thus, it is possible to design a barcoding strategy whereby the unique signature of a particular bead is based on a fluorescence number composed of, e.g., nine digits (i.e., the nine separate wavelengths), each digit able to assume 5 values (i.e., 0 through 4×). Combining these two variables yields a set of potential unique barcodes of 5⁹, or approximately 2 million different barcodes.

To stamp each bead with a barcode, a set of, e.g., 1×10¹³ beads is broken into one million groups of 1×10⁷ each. Each group of beads is placed in one well of a 384-well tray, requiring a total of about 2,600 trays. As one of skill will appreciate, this process may preferably be automated via known methods, using commercially available robotics. To the beads are added various quantities and types of fluorochrome dye such that the barcode requirements are fulfilled—i.e., that each type of bead has a unique barcode that will identify the associated ligand and distinguish it from all other ligands. The fluorochromes may readily be incorporated by dissolution in organic solvent followed by exposure to the beads for sufficient time to allow full diffusion and interaction with the beads. The organic solvent is then removed and the beads dried. Alternatively, various types of covalent chemical attachments to the beads may be employed, or the fluorescent dye may be incorporated into the bead by other methods known to the art, for example by synthesizing the beads from dye containing materials, or by encapsulating the fluorescent dye within the bead.

Generation of a Randomized Ligand Library for Screening

Once the beads are prepared with the desired fluorescent barcode or other such unique tag, the desired ligands (or secondary ligands) may be adhered to the beads, to form a series of uniquely tagged ligand sets.

A variety of methods for adhering a ligand to the support are known to the art, and one of ordinary skill can select a particular method based on the exact nature of the ligand to be adhered. For example, if the ligand is proteinaceous, the adhesion moiety may be, e.g., biotin/avidin, thioredoxin/phenyl arsine oxide, maltose binding protein/amylose, calmodulin/calmodulin binding peptide, dihydrofolate reductase/methotrexate, chitin/chitin binding protein, cellulose/cellulose binding protein or antibody/antibody epitopes such as the FLAG epitope, as described elsewhere herein. In each case, one binding moiety is expressed as part of a fusion construct in frame with the proteinaceous ligand, and the other is immobilized on the support by a covalent or noncovalent chemical linkage. In the case of hormones or other endogenous compounds, or other organic or inorganic molecules, the compounds may be attached via a chemical linker, e.g., a hydroxyl or primary amine, or may be synthesized directly on the bead.

If the ligand to be adhered is proteinaceous, then a subset of uniquely tagged, derivatized beads is exposed to a corresponding expression product lysate, which is collected in a particular location in, e.g., a 384 well array. The subset of identically tagged beads is suspended in solution and added to each well by either a pipetting device or by means of a magnetic dispenser (in the event that the beads are magnetic). The beads are mixed with the lysate in the well for a sufficient time to permit binding. This step thus generates subsets of uniquely identified ligands on randomizable supports.

It is most preferable to adhere each member ligand to its corresponding set of location-determinable supports in a substantially irreversible manner. Some adhesion moieties form such links by a covalent link or an extremely tight noncovalent link—e.g., the interaction between biotin and avidin, K_{d}=10⁻¹⁵ M. Such substantially irreversibly linked beads are ready for the next step in the process—exposure of the substrates to ligands that are firmly bound to their randomizable supports. However, if the interaction between the randomizable support and the ligand is reversible (e.g., on the order of K_{d}=10⁻⁶ to 10⁻¹⁰ M), an additional step may be employed. In this additional step, the ligands are eluted from the first set of supports (which may, in this instance, be unlabelled, as the various subsets of ligands at this juncture remain segregated) by addition of a large excess of soluble (i.e., unbound) ligand.

In the case of polypeptide/adhesion moiety fusion constructs, one adds an excess soluble adhesion moiety so as to competitively interfere with the interaction between the bead and the adhesion domain of the fusion construct, thus displacing the fusion construct from the bead. The soluble fusion construct then is re-attached via an irreversible linkage to another set of beads that are added to the solution in a location-determinable manner. This interaction may involve, e.g., binding avidin-coated beads by biotinylated fusion protein, or it may involve nonspecific, hydrophobic adsorption of the soluble protein onto the bead surface. Alternatively, it may be preferable to crosslink polypeptides to beads using, e.g., UV light of a specific wavelength and/or a chemical cross-linking agent, as is the case with the randomizable supports, described elsewhere herein.

Once all subsets of uniquely tagged beads have been successfully linked to the corresponding ligand subsets, then all the ligand subsets are collected by either a pipetting device or by the magnetic instrument and mixed into one integrated pool such that, e.g., all 1×10¹³ ligand-labeled beads are present. This step thus disperses all the tagged ligands into a fully randomized pool that represents all of, e.g., the one million protein-bead types, each type represented 10⁷ times. Each bead in the aliquot bears a ligand and a corresponding unique tag to identify that ligand. An aliquot of, e.g., 10⁷ beads is then drawn from this integrated pool of ligand-bearing beads. Each aliquot contains a statistically representative portion of the fully integrated ligand pool—i.e., a subset of beads representing a substantially full spectrum of available ligands (the degree of complete representation in any selected aliquot is determined by statistical sampling issues familiar to those in the art). Each location in the substrate array receives one aliquot of integrated ligand beads. Thus each arrayed substrate has the opportunity to interact with every ligand.

Preparation of a Location-determinable Support and Exposure to Substrates

Alternatively, in some embodiments of the invention, the substrates are adhered to a location-determinable support prior to exposure to the aliquots of integrated ligand-bearing supports. Generally, the two major characteristics of the location-determinable support are that (i) it is capable of adhering to the selected library polypeptide or other such substrate, and (ii) it is kept segregated so that it links the adhered substrate to the original clone array position (i,e., well) from which that substrate was derived. This support can be a fixed type of support, for example a finger, pin or other such probe that is rigidly arrayed so as to match the clone array (e.g., a 384 pin hand). Alternatively, the support can be a bead or other such microparticle, which is kept segregated in an array that directly correlates back to the original location in the substrate array (e.g., a set of beads that is kept segregated in one well of a 384 well tray, corresponding to the well of the 384 well tray from which, e.g., the original clonal polypeptide was derived). Microparticles may be preferable for selections that involve large numbers of substrate-ligand interactions, or that involve relatively specific or slow-forming interactions. Fixed supports offer advantages for reduced handling and/or automation.

As described above, it is most preferable that the substrate be linked in a substantially irreversible manner to the location-determinable support. If this is not accomplished by the initial adhesion step, then the substrates are eluted from the first set of supports by addition of a large excess of soluble (i.e., unbound) substrate. The substrate is then re-adhered to a second set of location-determinable supports in a substantially irreversible manner, as described above.

Exposure of each Substrate to the Integrated Ligand Library

Generally, this step requires that each uniquely located substrate (either in solution or adhered to its analogous location-determinable support) is exposed to an aliquot of integrated ligand-bearing supports. Typically, these ligands will be in an appropriate buffer that mimics conditions inside the cell (i.e., reducing environment, neutral pH, 150 mM salt), and can be added directly to each array location containing a corresponding soluble or bound substrate. The lysate buffer may be of the same makeup. The binding buffer also may have other additives, e.g., those designed to minimize non-specific binding (e.g., detergent, bovine serum albumin). If a fixed type of location-determinable support (e.g. a pin or finger) is used, it may simply be dipped into a well containing an aliquot of the randomized ligand-bearing supports. If the location-determinable support is a bead or other such microparticle, a set of such beads containing one particular substrate may be added to a well that contains a randomized aliquot of the ligand-bearing beads, and the two sets of beads mixed thoroughly so as to maximize substrate-ligand exposure. Interaction between the substrate and any of the many different ligands thus results in the corresponding ligand-bearing bead (with its unique identification tag) adhering to the substrate, thereby forming a bead-bead aggregate.

In some embodiments utilizing microparticles as location-determinable supports, it may be desirable to replace the support-bound substrate with soluble substrate after exposure to the ligand aliquots (and formation of substrate-ligand bead aggregates). In such cases, soluble substrates (termed herein, “replacement substrates”) are added to each array location that contains the corresponding bead aggregates. For example, in the case of individual or library polypeptides, the polypeptide domains of the replacement polypeptides are identical to those of the polypeptides bound to the supports. Because the replacement polypeptides are in vast excess, and because the interactions between polypeptides and ligands in solution are generally characterized by relatively rapid off-rates, the soluble replacement polypeptides bind the ligands and displace competitively the support-bound polypeptides. Thus, in a single step the location-determinable supports are displaced from the ligand-bearing randomizable supports and soluble replacement polypeptides are attached to the ligand-bearing supports in preparation for further characterization or screening. For example, in embodiments in which both the replacement substrate and the ligand are proteinaceous, the pairs may be subsequently exposed to secondary ligands, typically small organic molecules, as described herein. Small organic molecules that bind to the primary ligand, for example, can displace the replacement substrate, thereby identifying small a organic molecule with potential therapeutic value as a disruptor of a protein-protein interaction.

Alternatively, it may be preferable to detach the location-determinable supports in a separate step, followed by incubation of the segregated sets of interacting ligand-bearing beads with soluble replacement polypeptide or such substrate. This may be accomplished, for example, by hyrolysis of a linker that attaches the library polypeptides to the location-determinable supports. If a DNA linker is used, DNAse treatment may release the location-determinable beads, while the residual fusion protein remains bound by noncovalent forces to the ligands on the randomizable beads. A second binding step involving the ligand-bearing beads and soluble replacement polypeptides is then performed in order to adhere the second layer (the library polypeptide layer) to the bead prior to detection of polypeptide-ligand complexes. This replacement step is generally applicable to non-proteinaceous substrates, as well.

Magnetic Interactions

In one embodiment of the invention, beads formed from a magnetic resin are used as the location-determinable support. In this embodiment, a set of magnetic beads (e.g., 10⁷ beads per well) is apportioned into each array location, which contains a corresponding library polypeptide or other such substrate. As the magnetic beads have adhesion domain binding moieties that are complementary to those of, e.g., the fusion polypeptides conjugated to their surfaces, after some period of time saturating or near-saturating amounts of fusion protein will adhere to the resin, and the polypeptide-coated beads are collected. This may be accomplished by dipping a magnetic pin into each well, allowing the magnetic beads (with the adhered substrates) to be drawn to the pin, withdrawing the beads, transferring to another well, and discharging the magnetic bead by demagnetizing the pin. In other embodiments, the magnetic forces may be applied externally to pull the magnetic beads to the well wall, with subsequent removal of the remaining non-magnetic materials.

Next, substrate/ligand bead aggregates are formed and collected. First, each set of magnetic beads in the array is exposed to aliquots of non-magnetic ligand-bearing supports. After a period of time to permit interactions between substrates and ligands, the magnetized beads are again collected with the aid of a magnetic device. Any of the ligand-bearing beads that have interacted to form aggregates with the magnetized beads are pulled along with the magnetic beads to the magnet. Ligand-bearing beads that do not interact are left behind in solution. The aggregates of magnetic beads and interacting ligand-bearing beads are then collected. Thus, only those beads that contain interacting substrates and ligands are recovered for subsequent quantitative analysis.

Conversely, the ligand-bearing randomizable supports may be magnetized while the location-determinable supports remain unmagnetized. The magnetized randomizable supports then function analogously to gather the bead aggregates formed by the substrate/ligand complexes.

In using magnetic forces to cull out interacting substrate/ligand complexes, a “surface interaction” as opposed to solution interaction is created, and provides an enrichment for substrate-ligand interactions. This enrichment step obviates the need to examine carefully every possible substrate-ligand interaction using a quantitative, but serial device such as a flow cytometer. Accordingly, interaction sets on the order of 10⁶×10⁶ polypeptides (akin to a human protein interaction map) may be screened rapidly and efficiently by inserting a bead-bead interaction step.

Segregating, Identifying and Quantifying the Substrate/Ligand Pairs

Once the substrate/ligand interactions are consummated, the interactions can be quantified, and each substrate and ligand identified as follows.

In the case of proteinaceous substrates, one ultimately obtains a set of supports that bear a polypeptide layer reversibly bound to ligand-bearing randomizable supports (i.e., either the randomizable supports were exposed only to soluble polypeptides, or the bead-bound polypeptides were subsequently displaced by an intervening exposure to soluble polypeptides). Such polypeptide/ligand complexes may be rapidly quantified by use of a fluorescence-activated cell sorter. The fluorescent signals emitted by the unique tags on the ligand-bearing supports provide the basis for rapid and accurate quantitation by this method.

In other embodiments, substrate-ligand complexes can be detected by either detecting a unique recognition domain (e.g., epitope) on the polypeptide or ligand (by “unique” is meant either that the recognition domain exists on only one member of the complex, or alternatively that it is present on both members but sterically accessible only on the outer layer). Supports that bear a ligand may be identified by a variety of immunological or fluorescence techniques known to those in the art. As one non-limiting example of such identification, a fluorescence-labeled antibody that reacts with such an epitope on the library polypeptide is utilized. After a period of time suitable for antibody binding (typically one half hour), the beads are collected and examined by an instrument such as a FACS machine to measure the level of antibody (determined from the fluorescence signal of the particular fluorochrome attached to the antibody). Concurrently, the randomizable support barcode can be read by fluorescence measurements at other wavelengths. This in turn reveals the identity of the fusion protein attached irreversibly to the randomizable support. The identity of the soluble protein is retained based on the well from which the bead was collected (i.e. the unique array location) immediately prior to the detection step. Thus, both the identity of the primary, irreversibly attached protein and the soluble protein is known, and the approximate strength of the interaction between them can be determined from the antibody fluorescence signal.

For some applications, a CCD camera may be utilized to detect interacting substrate-ligand complexes. For example, in applications screening for interaction of a non-proteinaceous organic molecule with a polypeptide, a CCD system can be used to visualize interacting complexes, thereby providing both detection and quantification. The CCD camera can detect a variety of visual outputs, including without limitation fluorescent emissions, chemiluminescent emissions, and SPA (scintillation Proximity Assay) emissions. In the SPA format, one member of the interacting pair is radiolabeled using standard techniques, and the other member of the pair is adhered to a bead in which a radio-detecting scintillation component is incorporated in the interior of the bead. When the radiolabeled component interacts with the bead-bound component, a detectable scintillation signal is emitted. The beads can optionally be displayed on some surface, for example an identification grid with grid locations correlating to each unique array location, for scanning by the detector.

One non-limiting example of CCD detection of fluorescent signals utilizes a scientific grade CCD camera incorporating a high quantum efficiency image sensor. The target molecules are distributed along the well bottoms of optically transparent microtiter plates. The CCD, fitted with lenses and optical filters, acquires images of the through the optically transparent well bottoms. Fluorescent excitation of the fluorescent molecules is generated by appropriately filtered coherent or incoherent light sources. The resulting digital images are stored on a computer for subsequent analysis.

An exemplary detection system is composed of a PixelVision SpectraVideo™ Series imaging camera (1100×330 back-illuminated array), PixelVision PixelView™ 3.03 software, two 50-mm/f1.0 Canon lenses, four 20750 Fostec light sources, four 8589 Fostec light lines, one 59345 Oriel 510-nm band pass filter, four 52650 Oriel 488-nm laser band pass filters, a 4457 Daedal stage, Polyfiltronic clear bottom microtiter plates, and supporting mechanical fixtures. Mechanical fixtures are constructed to position the PixelVision camera below a microtiter dish. Additionally, the fixtures mounted four Fostec light lines and allowed the excitation light to be focused on the viewed area of the microtiter dish. The two Canon lenses were butted up against each other front to front. A 510-nm filter is placed between the two lenses. The front-to-front lens configuration provides 1:1 magnification and close placement of the target object to the imaging system.

The above-described techniques quantify polypeptide binding pairs or polypeptide/ligand binding pairs. Optionally, the exact make-up of each binding pair is ascertained by identifying (i) the unique array location from which the library polypeptide or other such substrate is derived, and (ii) the ligand identity that corresponds to the unique tag on the bead (which, in the case of creating protein interaction maps, will in turn relate back to another unique library polypeptide array location). Optionally, if sequence information about a given interacting polypeptide is desired, one may sequence the DNA encoding the polypeptide produced by each unique location in the library array.

### DESCRIPTION OF PREFERRED EMBODIMENTS

### EXAMPLE 1

### Lysate Libraries

Expression Vectors

In order to generate sufficient amounts of polypeptides for ligand screening, it is desirable to first clone DNA encoding the library polypeptides of interest into a vector that is suitable for high levels of expression of those polypeptides. The host cells of interest are transformed with such an expression vector, production of the library polypeptides is induced, and the library polypeptides are collected.

A variety of expression vectors are suitable for use in this invention. As one non-limiting example, an expression vector bearing an inducible trc promoter was used. Plasmid pSE420 (Invitrogen) features the trc promoter, the lacO operator and lacI^{q} repressor, a translation enhancer and ribosome binding site, and a multiple cloning site. For insertion into this vector, the E. coli thioredoxin gene was amplified from pTrx-2 (ATCC) in such a manner as to retain a restriction enzyme site on the 5′ side of the gene, and was cloned into the pSE420 vector's multiple cloning site at the 5′ NheI and 3′NgoMIV locations, thus placing it under control of the trc promoter. The thioredoxin gene can advantageously enhance recombinant protein solubility and stability. Moreover, as a cytoplasmic protein, it can be produced under reducing conditions but still can be released by osmotic shock because of accumulation at adhesion zones.

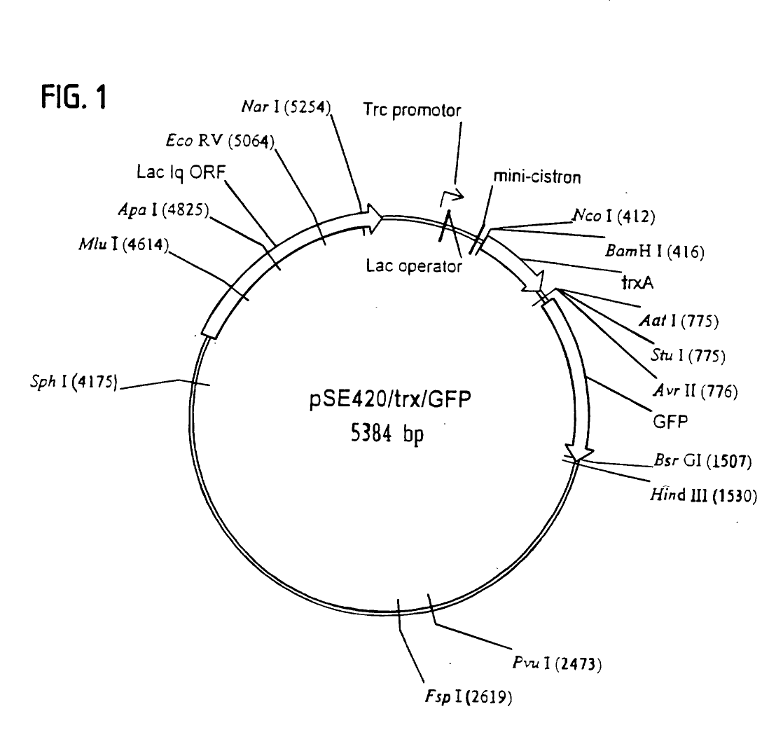

Once the pSE420 plasmid was modified to contain the thioredoxin gene (pSE420/trxA), the gene encoding GFP was inserted in frame with the thioredoxin, in order to rapidly isolate intact, in-frame constructs and thereby to eliminate constructs in which the library polypeptide would not be properly produced. The gene encoding EGFP was PCR amplified from plasmid pEGFP-1 (Clontech), maintaining a NotI restriction site 3′ of the EGFP sequence, and establishing a second NotI site 5′ of that sequence. The NotI sites may be used to readily remove the EGFP fragment from the vector after intact constructs are isolated. The NotI fragment containing EGFP was then cloned into the NotI site of the pSE420/trxA vector. Vectors containing the EGFP in frame and in the correct orientation were designated plasmid pSE420/trxA/EGFP. FIG. 1.

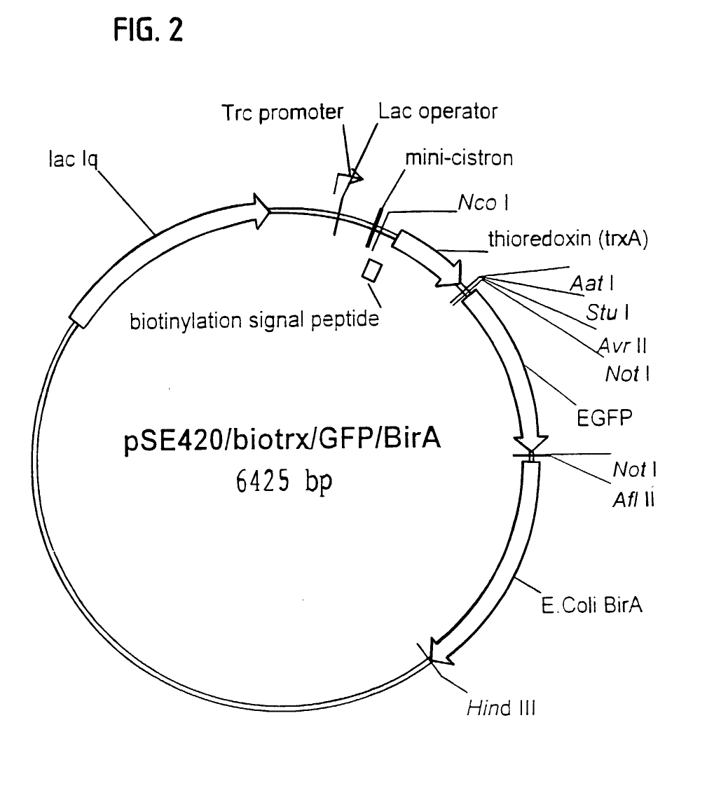

Once the vector containing the desired promoter and other components is prepared, DNA encoding the desired adhesion moiety is introduced. For example, a biotinylation signal may be used to adhere the library polypeptides to steptavidin beads. The in vivo biotinylation peptide sequence was cloned into the pSE420/trxA/EGFP vector (FIG. 1) in frame to the amino terminus of the thioredoxin gene by cutting at the 5′ NcoI and 3′ NheI site and filling in the overhanging nucleotides with Klenow prior to ligation. The biotinylation signal peptide is 23 residues long (Tsao et al, Gene 169:59-64 (1996)), and the sequence that encodes it can be readily synthesized on an oligonucleotide synthesizer using standard techniques. The vector may advantageously be modified to include the BirA gene, which encodes the enzyme responsible for adding biotin to the recombinant biotinylation signal. The BirA gene was amplified from genomic E. coli DNA by PCR. A copy of the BirA gene was added in a polycistronic fashion to the carboxyl terminus of the biotin/trxA/EGFP sequence and the resultant modified pSE420 vector was designated pSE420/biotrx/GFP/BirA (FIG. 2).

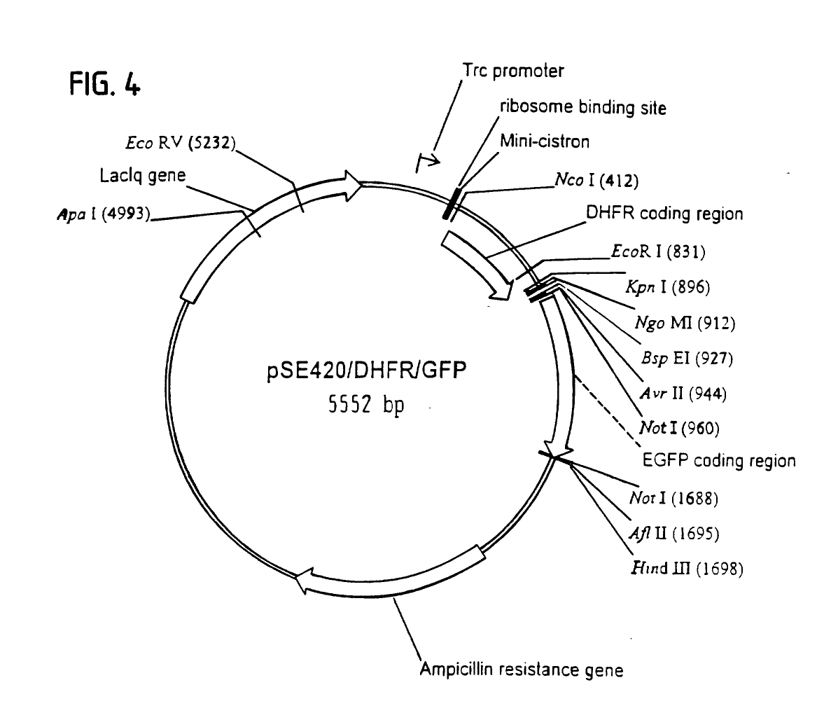

An alternative adhesion moiety, dihydrofolate reductase (DHFR) was incorporated into the expression construct as follows. The DHFR gene was amplified from E. coli genomic DNA by PCR with NcoI and KpnI sites on the 5′ and 3′ ends, respectively. This fragment was cloned into the NcoI/KpnI site of pSE420. Subsequently, the NotI fragment containing EGFP (described above) was cloned in frame with DHFR into the NotI site. The resultant plasmid was designated pSE420/DHFR/GFP (FIG. 4).

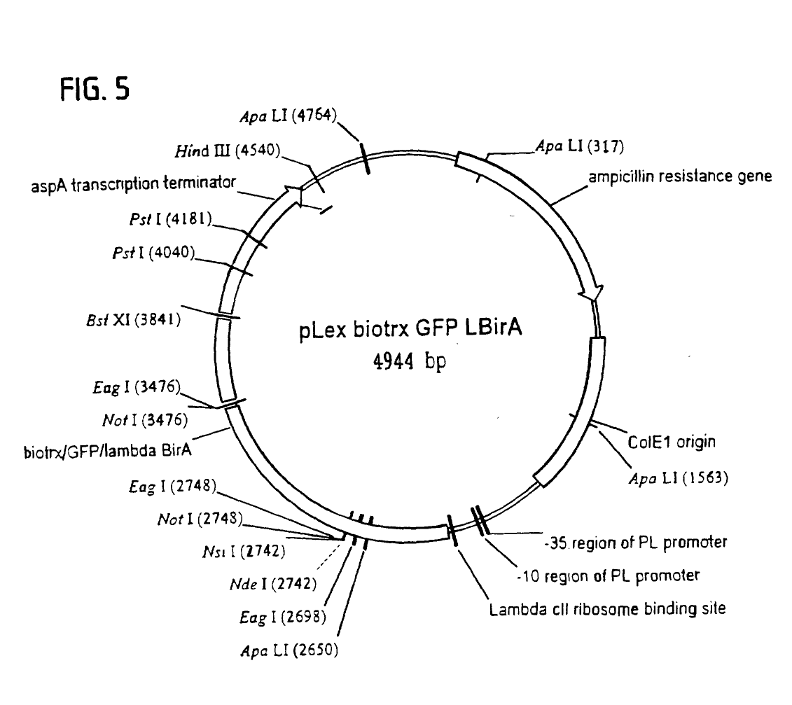

Another promoter system suitable for use in the invention features the P_{L} promoter. This system was constructed by digesting the pLex plasmid (Invitrogen) with NdeI and PstI and blunting the resultant ends with mung bean nuclease. The pSE420/biotrxGFP/BirA construct described above was digested with NcoI and HindIII, and the NcoI/HindIII fragment then blunt-ended with T4 polymerase. This fragment was then inserted into the pLex construct. The resulting plasmid was designated pLex/biotrx/GFP/BirA (FIG. 5). Optionally, the DHFR/GFP expression cassette described above may be inserted into the pLex plasmid by digesting pLex with NdeI and PstI, blunting the ends with mung bean nuclease, and inserting the blunte-ended NcoI/HindIII fragment from pSE420,DHFR/GFP.

Following construction of the described vectors, expression was induced by introduction of the appropriate induction agent (IPTG for pSE420-based expression vectors, and tryptophan for pLex-based vectors). Production of the recombinant polypeptide insert was detected by GFP fluorescence via FACS, or by western blot analysis. The recombinant polypeptides were then selectively bound and removed from bacterial lysatyes of induced cultures via binding with the respective binding partner (streptavidin for biotrx/GFP and methotrexate for DHFR/GFP), which had been immobilized to beads, as described elsewhere herein.

Library Polypeptides

DNA encoding the library polypeptides may be derived from a variety of sources, using techniques that are familiar to the art. As one non-limiting example, a cDNA library encoding human protein domains was prepared, using methods that are well known in the art, from human placental tissue. Poly(A) RNA was isolated from placental tissue by standard methods. First strand cDNA was then generated from poly(A) mRNA using a primer containing a random 9 mer, a SfiI restriction endonuclease site and a site for PCR amplification (5′-ACTCTGGACTAGGCAGGTTCAGTGGCCATTATGGCCNNNNNNNNN). The second strand was then generated using a primer consisting of a random 6 mer, another SfiI site, and a site for PCR amplification (5′-AAGCAGTGGTGTCAACGCAGTGAGGCCGAGGCGGCCNNNNNN). After conducting a number of PCR amplification cycles, the DNA was cut with SfiI and the resultant fragments were size-selected for fragments of greater than about 400 bp. The selected fragments were ligated into the Sfil sites of a suitable expression vector, as described herein. The library polypeptide DNA fragments then were isolated and inserted in frame with DNA encoding a corresponding biotin adhesion moiety and thioredoxin. DNA encoding the library polypeptides was prepared by cutting the DNA with SfiI and then inserted at an SfiI site placed in a linker (5′ GGCCGAGGCGGCCTGATTAACGATGGCCATAATGGCC) placed at the NgoMIV-AvrII sites of plasmid vector pSE420/biotrx/GFP/BirA, or of plasmid vector pET-biotrx-GFP-BirA.

To select for those cDNAs that are in-frame with TrxA, E. coli expressing constructs possessing in-frame cDNAs are selected by FACS sorting and selecting for bright (i.e., “green”) cells. Such cells are expressing intact GFP, which is in frame with and downstream from the library polypeptide and TrxA sequences. Plasmid DNA is isolated and the EGFP insert then removed via NotI digestion. Once the EGFP marker has been used to sort cells and removed from the modified pSE420 vector, the modified pSE420 plasmids are again transformed into E. coli and expressed via IPTG induction.

Other Adhesion Moieties

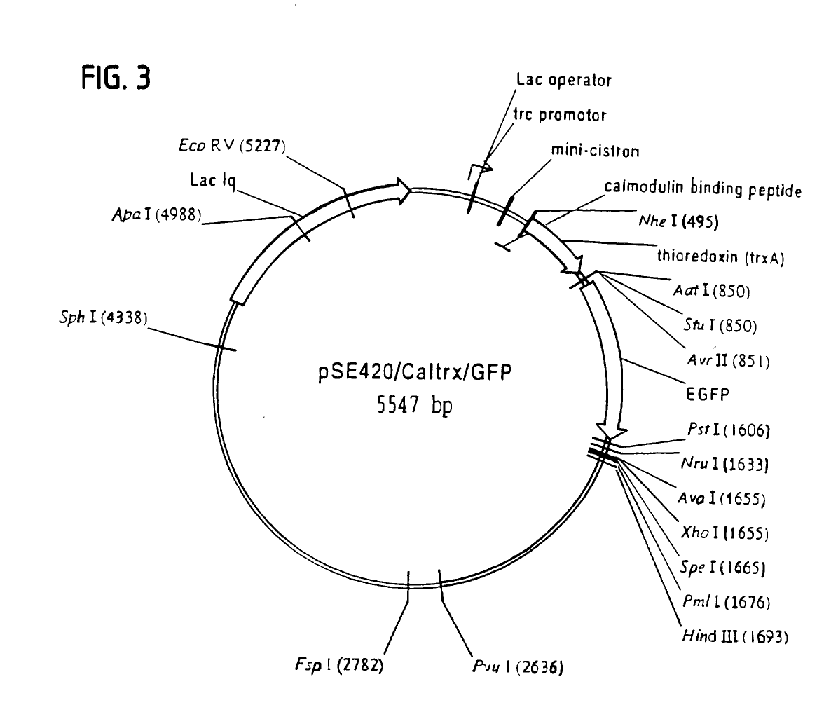

Alternatively, the library polypeptides may adhere to calmodulin-containing beads using calmodulin binding peptide (“CBP”) as the adhesion moiety. The vector constructs are prepared as described above, but an expression cassette containing CBP is inserted into the vector immediately 5′ of the trxA gene via the 5′ NcoI and 3′ NheI sites, as described above. FIG. 3. The CBP thus is used in place of the biotinylation signal peptide, and immobilizes the library polypeptides to the calmodulin beads.

As another alternative to the above-described system, the thioredoxin gene product may itself serve as the adhesion moiety, and will bind the fused library polypeptides to phenylarsine oxide (“PAO”) beads. Polystyrene beads are modified so as to covalently link phenylarsine oxide to the surface by reacting the carboxyl groups on the bead surface with p-aminophenylarsine oxide via a water soluble carbodiimide. Kaleef and Gitler, Methods of Enzymology 233:395-403 (1994). The above-described pSE420/trxA/EGFP vector in this instance is used directly, i.e., no subsequent moiety is fused to the carboxyl terminus of the thioredoxin gene. Screening and expression are carried out as described above.

As still another alternative, the library polypeptides may simply be adhered to polystyrene beads via hydrophobic adsorption. In such embodiments, the library polypeptides are first separated from, e.g., the host cell polypeptides by standard methods before exposure to the beads.

Crosslinked Embodiments

In some embodiments, polypeptide substrates or ligands may be crosslinked with the supports. As one non-limiting example, the bacterial lysate containing the expressed recombinant fusion protein is incubated with microspheres containing a ligand specific for the fusion partner. Following binding of the fusion protein, a photoactive crosslinker on the microsphere will irreversibly bind the fusion protein. Examples of possible ligand-fusion partner combinations are, but not limited to, phenylarsine oxide (PAO) and thioredoxin (Methods of Enzymology (1994) 233, 395-403), or a suicide substrate and its corresponding enzyme (e.g. clavulanic acid and beta-lactamase; J. Mol. Biol. (1994) 237, 415-422).

In embodiments utilizing PAO and thioredoxin, the thioredoxin fusion product is constructed as described above. The PAO moiety, 4-aminophenylarsine oxide, is synthesized as described in the literature (Biochemistry (1978) 17, 2189-2192). The 4-aminophenylarsine oxide is then reacted with a large molar excess of BS₃ (Pierce Chemical Co.) in order to place an amine reactive NHS ester and 8 carbon spacer at the 4 position of 4-aminophenylarsine oxide. The NHS ester-modified PAO is then reacted in equimolar amounts with sulfo-SANPAH (Pierce Chemical Company) and 10 μm amine-functionalized latex microspheres (Polysciences, Inc.). The result of this reaction yields microspheres with approximately one-half of the available amine groups with PAO attached, while the remaining half have the photoactivatable crosslinker. These microspheres are then reacted with the bacterial lysate containing the expressed fusion protein. Vicinal dithiol-containing proteins, including the recombinant thioredoxin fusion protein, is bound to the microspheres. After washing steps to remove non-specifically bound proteins, the microspheres with the bound recombinant fusion protein are crosslinked to the microspheres via amine groups on thioredoxin by exposing to light at 320 nm-350 nm. These microspheres are then ready to be used as described elsewhere in this application.

In another non-limiting embodiment, library polypeptides are covalently attached to the supports by adsorption to the support, followed by crosslinking. For example, the library polypeptides may be constituted as fusions with maltose binding protein. These fusion constructs then are purified from the lysate using a maltose affinity resin and released with soluble maltose (J. Chrom. 633 (1993) p.273-280). The purified fusion constructs then are adsorbed onto polystyrene beads, thus attaching via hydrophobic interactions. Finally, the polypeptides are crosslinked with a phototactivated crosslinker, for example sulfo-SANPAH (Pierce Chemical Co.).

In yet another non-limiting embodiment, polypeptide substrates are attached to microparticles via the interaction of a DNA-binding protein and a DNA moiety or analog on a bead. Specifically, a DNA binding fusion library such as a Gal4 fusion is constructed. The corresponding microparticles have two features—a peptide nucleic acid (PNA) oligomer for binding the protein of interest, and a photoactivatable crosslinker, e.g. sulfo-SANPAH (Pierce Chemical Company), attached to the end of the oligomer. The microparticles are placed into lysates containing the various Gal4/library polypeptide fusion constructs, and those constructs then bind to the beads via interaction between the Gal4 binding moiety and the bead oligomer. The crosslinker is then photoactivated, thus forming the covalent linkage between the proteins and the beads.

Alternatively, the bacterial lysate containing the expressed recombinant fusion polypeptides are incubated with microspheres that bear a ligand specific for the fusion polypeptide. After the polyeptides bind to the beads via the ligands, a photoreactive crosslinker on the bead is activated so as to irreversibly bind the fusion polypeptide to the bead. Non-limiting examples of fusion polypeptide/ligand partners include DHFR/methotrexate, PAO/thioredoxin, or a suicide substrate and corresponding enzyme (e.g., clavulanic acid and beta-lactamase; J. Mol. Biol. (1994) 237:415-422).

For an embodiment utilizing the thioredoxin construct described elsewhere herein, 4-aminophenylarsine oxide is synthesized as described in the literature (Biochemistry (1978) 17:2189-2192), reacting the 4-aminophenylarsine oxide with a large molar excess of BS³ (Pierce Chem. Co.) in order to place an anime reactive NHS ester and and eight carbon spacer at the 4 position of the 4-aminophenylarsine oxide. The NHS-modified PAO is then reacted in equimolar amounts with sulfo-SANPAH (Pierce Chem. Co.) and 10 μm amine-functionalized latex microspheres (Polysciences, Inc.), yielding microspheres with approximately one half of the available amine groups with PAO attached, while the remaining half attaches the photoactibatable crosslinker. The microspheres are then reacted with the bacterial lysate containing the expressed thioredoxin fusion protein. Vicinal dithiol containing polypeptides, including the recombinant thioredoxin fusion protein, are thus bound to the microspheres. After washing steps to remove the non-specifically bound protein, the microspheres with the bound recombinant fusion polypeptide are crosslinked via the thioredoxin amine groups by exposing the complexes to 320-350 nm light.

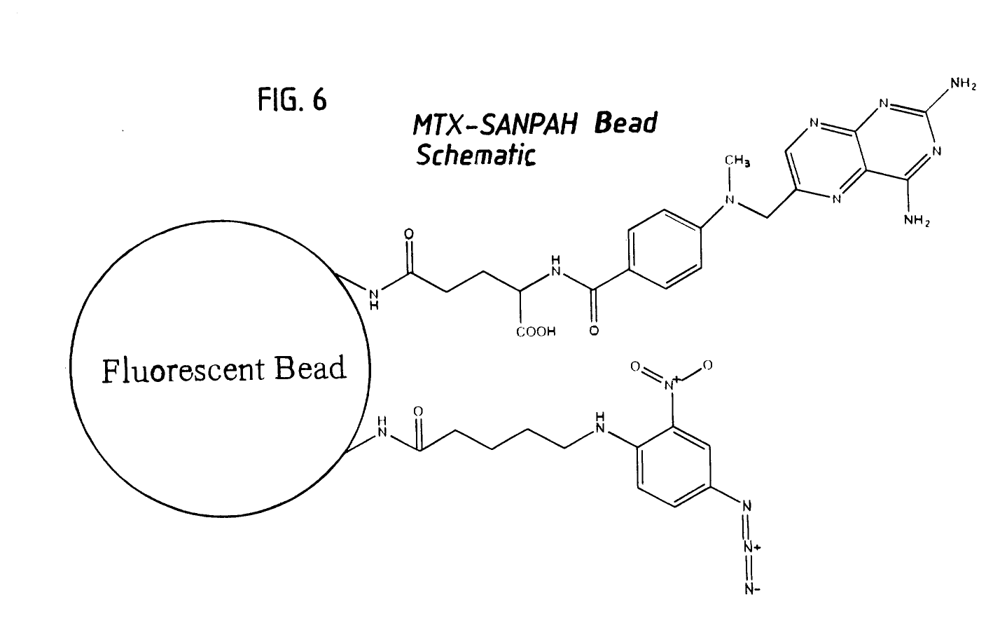

For a DHFR//methotrexate embodiment, the DHFR expression vector is as described elsewhere herein. The corresponding affinity resin, sulfo-SANPAH (Pierce Chem. Co.) is reacted with the amine-functionalized latex microspheres (Polysciences Inc.) in non-saturating amounts to couple the crosslinker onto the microspheres in non-saturating amounts. Methotrexate (Sigma Chem. Co.) is then reacted with EDC (Pierce Chem. Co.) and the sulfo-SANPAH functionalized beads so as to couple the methotrexate to available amine groups on the beads. The resultant functionalized microspheres are depicted in FIG. 6. A bacterial lysate containing DHFR fusion polypeptide is then bound and photo-crosslinked as described for the thioredoxin/PAO system.

In embodiments that utilize fluorescent identification tags, it may be preferable to first protect the fluorescent tags before undertaking chemical cross-linking. This may be accomplished in a variety of ways familiar to the art, including without limitation embedding the fluorescent tags beneath the surface of the bead, or chemically protecting the fluorescent tags by first derivatizing with non-reactive functional groups, and then de-protecting the tags once chemical crosslinking is complete.

Host Cells

A variety of host cells are suitable for use in this invention. One common species of host cell with utility here is E. coli. Preferred strains of E. coli are characterized by (1) over-expressing the necessary amount of protein required to fulfill other parts of the invention (coating of the beads, etc.), (2) tolerating “leaky” expression of toxic target plasmids, and (3) being amenable to cell lysis and protein recovery. Such strains include, without limitation, TOP10 (Invitrogen Corporation), BL21 (Novagen), and AD494 (Novagen). One such strain, BL21 (DE3) RIL (Stratagene), was selected for further study in this non-limiting Example.

These host cell strains are used in the presence or absence of the T7 phage gene encoding lysozyme which resides on the plasmid pLysS (Novagen). T7 lysozyme cuts a specific bond in the peptidoglycan cell wall of E. coli. High levels of expression of T7 lysozyme can be tolerated by E. coli since the protein is unable to pass through the inner membrane to reach the peptidoglycan cell wall. Mild lytic treatments of cells expressing T7 lysozyme that disrupt the inner membrane results in the rapid lysis of these cells. Thus, use of the pLysS plasmid should facilitate the lysis of E. coli host cells expressing the library polypeptide constructs.

Arraying Single-cell Clones

Prior to induction of fusion polypeptides, individual clones are arrayed at unique locations. The location from which each library polypeptide is derived will serve to identify it during subsequent screening steps. Each unique location is tracked throughout the screening, either by directly moving each segregated library polypeptide sequentially to other, correspondingly unique locations, or by indirectly tracking the origin of each library polypeptide via its corresponding location-determinable support, which is adhered to the library polypeptide via the adhesion moiety that was incorporated in the above-described fusion construct.

Methods for generating single-cell clones are known to the art. For example, the library is first plated to permit well-isolated colonies to grow. Cells from individual colonies may be isolated manually or via automated techniques such a colony picker, and cells from each isolated colony are placed at its corresponding unique location to generate a single-cell clone. Commercially available microtiter trays, for example in 96 or 384 well formats, provide convenient arrays for generating and tracking a unique location for each such single-cell clone. Alternatively, as described in more detail below, the process may be automated for generating arrays with large numbers of single cell-type clones, each of which generates a correspondingly unique library polypeptide.

Lysing the Host Cells

Following induction and expression, the host cells are harvested and lysed and the polypeptide-bearing lysate collected. A variety of lysing techniques are suitable for use in this invention, including without limitation the three techniques described in detail below. The cells also may be sonicated, for example with the use of commercially available sonicators designed for use with, e.g., 96 well plates (e.g., Misonix Incorporated. Model 431-T).

In one embodiment, host cells are lysed using osmotic shock. This technique is a simple method of preparing the periplasmic fraction of expressed proteins. In E. coli strains containing the pLysS plasmid, standard osmotic shock techniques can be modified as follows: T7 lysozyme-containing host cells are resuspended in ice-cold 20% sucrose, 2.5 mM EDTA, 50 mM Tris-HCl pH 8.0 to a concentration of OD₅₅₀=5 and incubated on ice for 10 minutes. The cells are centrifuged at 15,000×g for 30 seconds, the supernatant discarded, and the pellet resuspended in the same volume of ice-cold 2.5 mM EDTA, 20 mM Tris-HCl pH 8.0 and incubated on ice for 10 minutes. The cells are centrifuged at 15,000×g for 10 minutes. The supernatant contains protein fraction released due to osmotic shock. Total protein is assessed using the BCA Protein Assay kit.

In another embodiment, the host cells are lysed by employing a freeze/thaw protocol. This technique is intended for cells containing the pLysS plasmid. Such cells are resuspended in 1/10 culture volume of 50 mM Tris-HCl pH 8.0, 2.5 mM EDTA. The cells are frozen at −80° C. and then rapidly thawed in order to lyse the cells. The cell debris are pelleted at 15,000×g for 10 minutes and the supernatant saved. To shear the DNA, a DNA nuclease solution is added and incubated for 15-30 minutes at 30° C. The number of freeze/thaw cycles required is determined by monitoring lysate protein concentration.

In yet another embodiment, the host cells are lysed by addition of a mild detergent. This technique is also intended for cells containing the pLysS plasmid. Host cells lacking the pLysS plasmid were resuspended in 1/10 culture volume of 50 mM Tris-HCl pH 8.0, 2.0 mM EDTA and 100 μg/ml lysozyme. Cells were then incubated for 15 minutes at 30° C. Triton X-100 was added to a final concentration of 0.1% and incubated for 15 minutes at room temperature. The cell debris were pelleted at 15,000×g for 10 minutes and the supernatant saved. To shear the DNA, a DNA nuclease solution is added and incubated for 15-30 minutes at 30° C.

### EXAMPLE 2

### Preparation of Microbeads

A variety of supports can be used as randomizable supports for binding ligands, and location-determinable supports for binding the library polypeptides. Suitable supports include beads in a variety of sizes and compositions. Selection of a particular bead depends in part upon the type of adhesion to be used (i.e., chemical/covalent linking, or linking through biological adhesion moieties), and the size and type of library polypeptide or other ligand to be adhered to the bead.

One preferred system uses polystyrene microparticles of, e.g., 10 μm, to adsorb proteins onto the surface of the bead (Polysciences, Inc. or Bangs Laboratories, Inc.). Library polypeptides are adhered to such supports by hydrophobic interactions between the library polypeptides and the bead surface. Other ligands are adhered by, e.g., synthesizing the combinatorial ligand library on the surface of the bead itself, or by incorporating a reactive functional group into the ligand structure, by which a covalent link is formed to the bead surface.

The polystyrene beads are exposed to, e.g., the individual library polypeptides uniquely located in the library arrays by suspending an aliquot of the beads in a buffer that is compatible with the chosen lysate solution (e.g., for mild detergent lysis, 1% Triton X-100 may be used) and pipetting aliquots into each 384 well format microtiter well. The beads are mixed by repetitive pipetting or by shaking the array plates to ensure maximal dispersion. The beads are left in for approximately 5-15 minutes to several hours, depending on the scope of the population to be screened, to ensure greater than approximately 70-100% maximal adhesion of the polypeptides to the microsupports. Exact conditions are optimized by routine testing familiar to one of ordinary skill in the art. The beads bearing the library polypeptides or other ligands then are removed, for example by vacuuming the soluble contents of each well through the base of a 384 well filter plate and then collecting the remaining coated beads, which are then utilized for interaction screening, as described below.

Another preferred embodiment utilizes streptavidin coated polystyrene beads to bind fusion proteins containing biotin. Such beads feature streptavidin molecules saturated to 1.8 mgs per gram of 10 μm polystyrene particle. To form such beads, streptavidin molecules (Pierce) are coupled to polystyrene beads having surface carboxyl reactive groups (Polysciences, Inc. or Bangs Laboratories, Inc.) using techniques familiar to those in the art. The particles are placed in the buffer 2-[N-morpholino]ethanesulfonic acid (MES). They are reacted with 1-ethyl-3-(3-dimethylaminopropyl)carbodiimide hydrochloride (EDC) (Pierce) and N-hydroxysuccinimide (NHS) (Pierce) to form an acyl amino ester. Alternatively, the particles are reacted with EDC to form an amine-reactive O-acylurea intermediate, which can then react with the free amine on a polypeptide to covalently link the polypeptide (e.g., streptavidin) to the bead surface. After washing with MES to remove excess reagents, excess streptavidin (e.g., 18 mgs per gram of bead) is added and the reaction mixed. The derivatized beads are then ready to bind biotin-bearing fusion polypeptides.

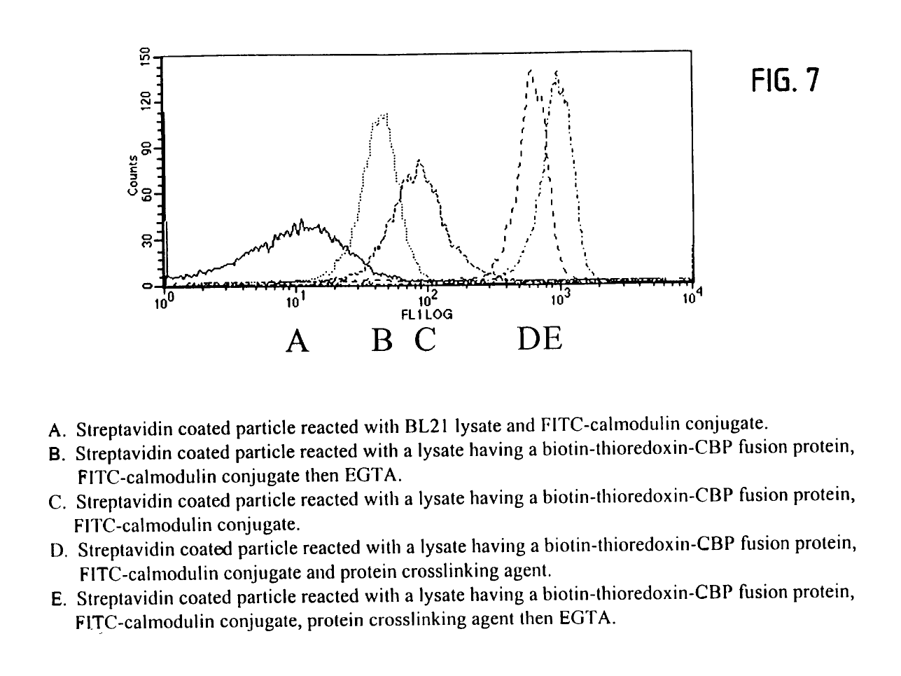

Still another preferred embodiment utilizes a calmodulin surface coating to bind fusion polypeptide constructs that include the calmodulin binding peptide (CBP). Such beads feature approximately 2.3 mgs calmodulin (Sigma) per 1 gram bead, covalently coupled to a 10 μm polystyrene particle, via the same chemistry described above for covalently linking streptavidin. In embodiments utilizing calmodulin and calmodulin binding protein (CBP), the moieties may be crosslinked as follows. A streptavidin coated 10 um particle (prepared as described above) was placed into a bacterial lysate in which a biotin-thioredoxin-CBP (biotrxCBP) fusion protein had been expressed, and the moieties allowed to bind. The beads were then washed to remove nonspecificly bound proteins, and reacted with a commercially available purified calmodulin having FITC covalently attached to the protein (Sigma). In the presence of calcium this interaction takes place (FIG. 7). Upon the removal of calcium, the calmodulin/CBP interactions begin to dissociate. However, when the CBP-calmodulin was reacted with a crosslinker such as disuccinimidyl suberate, the calmodulin/CBP interaction remained stable even in the absence of calcium.

Magnetic Beads.

In some embodiments, magnetic beads may be used to facilitate collection of the adhered polypeptides, ligands, or interacting pairs. One preferred embodiment of such a magnetic bead features a magnetic core with a polystyrene exterior coating, sized from 1-10 μm (commercially available Polysciences, Inc. or Bangs Laboratories, Inc). Such magnetic beads will bind proteins by direct adsorption, via the polystyrene coating. Alternatively, streptavidin-coated magnetic beads may be used. A variety of sizes are suitable, including 135 nm diameter beads (Immunicon, Inc.), 50 nm diameter beads (Miltenyi Biotec Inc.), 1 μm diameter beads (Bangs Laboratories), 2.8 μm diameter beads (Dynal Inc.) and 5 μm diameter beads (CPG Inc.). In still another embodiment, calmodulin coated magnetic particles are used. Such particles are synthesized by the same technique described above for streptavidin coated microparticles, but with the exception that calmodulin is substituted for streptavidin. Again, the starting particle is a magnetic particle with carboxy functional groups on the surface (Bangs Laboratories or Polysciences, Inc.).

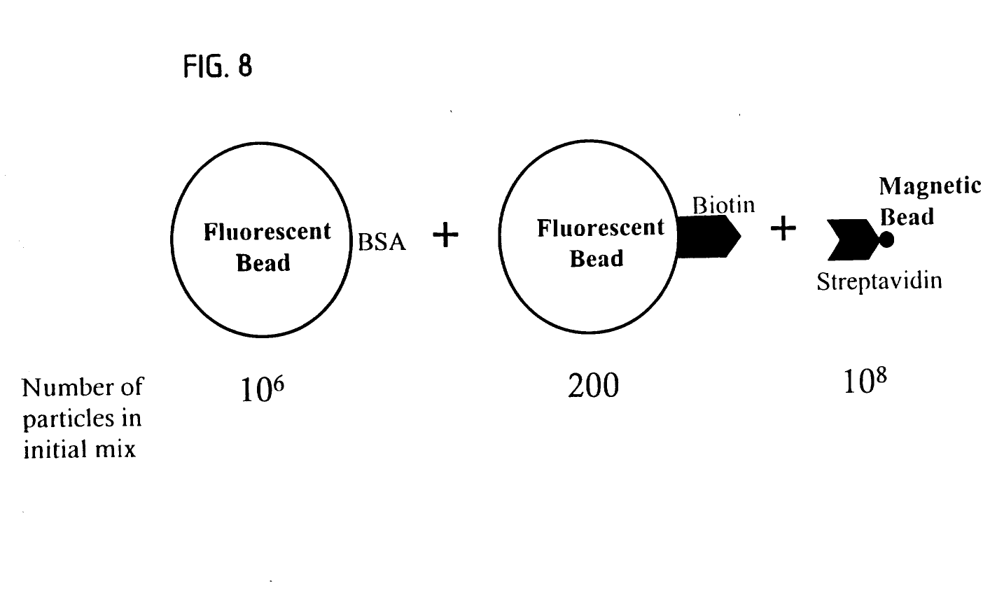

Interactions of a protein on a 10 um polystyrene bead and a protein on a 150 nm magnetic bead were carried out in two systems. In one system, one set of 10 um beads (prepared as described above) were coated with biotin, and another set of 150 nm magnetic beads (Immunicon) were coated with streptavidin. A reaction tube was set up with 10⁶ BSA coated 10 um beads, about 200 10 um biotin coated beads and about 10⁸ 150 nm streptavidin coated particles in PBS with 0.5% BSA. FIG. 8. These were reacted together for fifteen minutes to allow for binding between the biotin and streptavidin moieties. In order to enrich for these aggregates, a neodymium-iron-boron magnet was placed to the side of the tube and the liquid removed. After several washes with PBS the number of biotin coated and BSA coated particles were counted with a hemacytometer. It was found that the mixture had been enriched several thousand fold for the biotin coated particles.

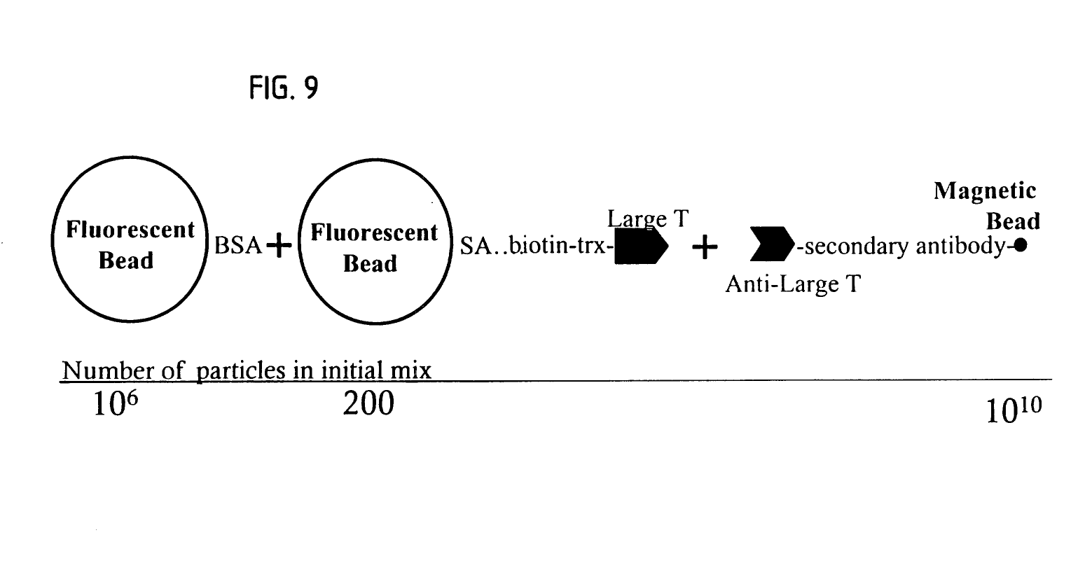

The other system examined the interaction of SV40 large T antigen with an antibody to the antigen. First, streptavidin coated 10 μm beads prepared as described elsewhere herein were added to a lysate containing a biotin thioredoxin SV40 large T antigen fusion protein (prepared as described elsewhere herein). About 200 of these large T antigen coated beads were added to a mixture of about 10⁶ BSA coated 10 um beads, along with some 10¹⁰ 150 nm magnetic beads coated with goat anti-mouse secondary antibodies (Immunicon), and 0.5 ug of mouse anti-SV40 large T antigen (Santa Cruz). FIG. 9. The reaction proceeded as above. Again the enrichment was several thousand fold for the 10 um SV40 large T antigen coated beads.

Fluorescence-tagged Beads.

In order to distinguish one type of ligand from another, each such ligand may be adhered to a randomizable support that bears a corresponding unique tag. One way of creating a unique tag is to adhere to the exterior surface of a nonporous randomizable support, or to entrap within interior regions of a porous randomizeable support, a particular mixture of fluorescent dyes—a unique fluorescent dye identifies, also referred to herein as a fluorescent “bar code”. The fluorescent dyes may be organic in nature, or alternatively may be fluorescent nanoparticles. Two variables contribute to the bar code—type of dye (i.e., its particular emission spectrum) and concentration of dye (i.e., intensity of its emission signal). A wide variety of fluorescent dyes with well-characterized excitation and emission spectra are commercially available. For example, Molecular Probes, Inc provides a variety of organic dyes; (see TABLE 1, below). Alternatively, fluorescent nanoparticles may be obtained that feature specific excitation and emission spectra. Such nanoparticles are described by Bruchez et al, Semiconductor nanocrystals as fluorescent biological labels, Science 281: (5385):2013-16 (September 1998) and Cahn, W. C. and Nie, S, Quantum dot bioconjugates for ultrasensitive nonisotopic detection, Science 281(5385):2016-18 (September 1998), the disclosures of which are incorporated herein in their entireties. Indeed, it is possible to procure sets of fluorescent molecules that cover the spectrum from blue to red. Each dye has characteristic excitation and emission spectra that may be used to create a bar code.

In one embodiment of the invention, a set of fluorescent bar codes is created that is sufficiently large to uniquely identify each member of a ligand pool on the order of 1×10⁶ members (i.e., roughly each protein encoded by a human cell). Optimally, the corresponding set of unique tags is generated from a set of 4-10 separate fluorescent dyes. The dyes are chosen so that there is optimal compatibility of their excitation and/or emission maxima when such dyes are irradiated by any one of a given FACS machine lasers, including Argon and Helium-Neon. The dyes are selected further so that there is minimal overlap of their emission maxima. Moreover, the dyes are chosen so as to be distinguished from any autofluorescence emissions of the bead to be labeled. However, as described below, it is possible to choose dyes that have some overlap because the dye cross-talk can be mathematically reduced or eliminated by certain computations that can be performed off line (i.e., by computers that use stored fluorescence data files as input).

TABLE 1
EXEMPLARY ORGANIC DYE SPECIES

| Molecular | | Excitation | Emission |
|---|---|---|---|
| Probes, Inc. | | wavelength | maxima |
| Catalog # | Dye Name | (nm) | (nm) |
| A-191 | 7-amino-4- | 351 | 430 |
| | methylcoumarin | | |
| B-3932 | bodipy ® 665/676 | 665 | 676 |
| C-652 | 5-(and-6)-1 | 599 | 667 |
| | carboxynaphtho | | |
| D-113 | dansyl cadaverine | 335 | 520 |
| D-275 | DiOC18 | 484 | 499 |
| D-282 | DiOC18(3) | 548 | 564 |
| D-307 | DiOC18(5) oil | 644 | 663 |
| D-2184 | Biodipy ® FL, SE | 488 | 530 |
| D-2186 | bodipy ® 530/550 | 530 | 550 |
| D-2187 | bodipy ® 530/550SE | 530 | 550 |
| D-2190 | bodipy ® 493/503 | 493 | 503 |
| D-2191 | bodipy ® 493/503SE | 493 | 503 |
| D-2219 | Bodipy ® 558/568, SE | 558 | 568 |
| D-2221 | bodipy ® 561/570 | 561 | 570 |
| D-2222 | bodipy ® 564/570SE | 564 | 570 |
| D-2225 | Bodipy ® 576/589 | 576 | 589 |
| D-2227 | bodipy ® 581/591 | 581 | 591 |
| D-2228 | Bodipy ® 581/591,SE | 581 | 591 |
| D-3921 | bodipy ® 505/515 | 505 | 515 |
| D-3922 | bodipy ® 493/503 | 493 | 503 |
| D-6102 | Biodipy ® FX-X, SE | 488 | 530 |
| D-6117 | Bodipy ® TMR-X, SE | 540 | 560 |
| D-6180 | Bodipy ® RGG, SE | 530 | 550 |
| D-6186 | Biodipy ® R6G-X, SE | 530 | 550 |
| | fluorescein | | |
| D-10000 | Bodipy ® 630/650- | 630 | 650 |
| D-10001 | Bodipy ® 650/665-X, SE | 650 | 665 |
| D-12731 | DiOC18(7) | 748 | 780 |
| N-1142 | nile red | 552 | 636 |

In other embodiments, fluorescent nanocrystals (Quantum Dot, Corp., Palo Alto Calif.) may be utilized as the fluorescent dye species forming the barcode. Briefly, the nanocrystal is a semiconductor material such as zinc sulfide-capped cadmium selenide. The nanocrystal also may feature an outer layer to aid in derivatization and/or to aid solubility, for example mercaptoacetic acid (Chan and Nie (1998), supra), or silica derivatives (Bruchez et al. (1998), supra. The emission spectrum of the nanocrystal is dependent upon the size of the cadmium selenide core of the crystal.

Fluorescent nanocrystals may be coupled with the beads in a variety of ways. One general approach is to apply absorption techniques such as are used in absorbing organic fluorochromes to beads. Briefly, the nanocrystals can be rendered nonpolar for this purpose by coating the nanocrystals with a nonpolar coating such as an alkyl silane. A polystyrene bead having a porous structure is then exposed to the nonpolar fluorescent nanocrystals, using methods familiar to those in the art. The nanocrystal then equilibrates into the corresponding nonpolar interior of the polystyrene bead, and is maintained there by repulsion from an aqueous solvent. Optionally, more porous particles (Dyno Particles, Inc.) may be utilized to increase the available interior region.

Alternatively, the nanocrystals may be linked to the selected beads via covalent bonds, using a variety of different chemistries familiar to those of skill in the art. In such embodiments both the bead surface and the nanocrystals are derivatized with surface reactive groups. In some embodiments, the bead features a porous surface, allowing the nanocrystals to diffuse into the interior regions of the bead prior to covalently cross-linking with the bead. In other embodiments, nonporous bead particles may be used, in which case the nanocrystal is crosslinked to the exterior surface of the bead.

A variety of beads and crosslinking chemistries are suitable for use in this invention. For example, in some instances it is advantageous to use porous silica particles having low autofluorescence. As one nonlimiting example, carboxyl coated silica particles (CPG, Inc.) of a desired size (e.g., 10 μm diameter) are selected. The nanocrystals are first reacted with an amine silane, thereby forming an amine functional group. The derivatized beads and nanocrystals are then mixed together so that the nanocrystals diffuse evenly throughout the particle. A crosslinking agent such as EDC (1-ethyl-3-(3-dimethylaminopropyl)carbodiimide) is then added, thereby conjugating the nanocrystal to the derivatized silica particle. In other embodiments, other derivatized particles may readily be substituted.

Fluorescence Barcoding.

The barcoding system uses a set of dye species chosen with the considerations enumerated above, as exemplified but not limited to those dyes in Table 1 or the nanocrystals described above. The identity of each randomizable support is encoded as a numerical readout having digit placeholders equal to the number of dyes used (e.g., nine dyes create nine “digits” in the barcode). Each digit in the barcode is then further defined by the amount of the specific dye, as determined from its fluorescence intensity (i.e., 0×, 1×, 2×, 3× or 4×). Thus, for 9 dyes and 5 amounts (or fluorescence levels) there are (5)⁹ possible barcodes.

The beads are labeled with dyes by mixing the selected number of dyes in defined ratios such that a specific bead receives a unique barcode. For example, using nine different dyes one defined bead type may receive dyes in the ratio of (4, 2, 3, 3, 1, 1, 2, 4, 2); a second bead type may receive dyes in the ratio (2, 2, 3, 3, 1, 1, 2, 4, 2). These beads differ only in the levels of the first dye (the first bead type has level 4, the second has level 2).

Fluorescent organic dye species may be selected from a wide variety of known dyes and incorporated into a wide variety of known beads, utilizing techniques familiar to those of skill in the art. E.g., U.S. Pat. No. 5,573,909, the disclosure of which is incorporated by reference herein in its entirety. As a non-limiting example, by mixing the dyes in an organic solvent such as, e.g., acetonitrile or dimethylformamide, and adding the dye solutions in defined ratios to individual groups of beads and allowing the absorption reactions to go to completion, it is possible to irreversibly adsorb dye molecules onto the bead surface and interior. Removal of the organic solvent followed by drying, leaves the beads labeled with the nine dyes in the predetermined amount dispersed over the surface of each bead. Fluorescently labeled beads prepared in this general way but with only one or a few fluorescent tags have been described in the literature (Michael et al., Analytical Chemistry 70(7):1242-48 (1998); Fulton et al., Clinical Chemistry 43(9):1749-56 (1997)) and are available commercially (Luminex Corp.).

As one non-limiting example of the barcoding strategy, four dyes were selected for study: BioDIPY 493N, BioDIPY 560PA, BioDIPY580PA and BioDIPY665N. The dyes were incorporated into polystyrene beads (Bangs Labs, Inc. PS07N) beads as follows. The selected dyes were dissolved in dimethylformamide (DMF). The beads were washed three times with absolute ethul alcohol (and stored in same). A staining mix was prepared, containing 10% DMF, 54% absolute ethyl alcohol and 36% dichloromethane (approximating a 60:40 ratio of ethyl alcohol to dichloromethane). The beads were added and rapidly stirred for ten minutes. The staining solution was then removed from the beads by centrifugation or filtration and the beads were washed two times with absolute methanol followed by two washes of PBS/1% TWEEN 20. The dyed beads were then stored in the PBS/TWEEN 20 mixture at 4° C., protected from light. The beads were doped with five different concentrations of each dye, as summarized below in Table 2.

TABLE 2
SUMMARY OF DYE PROFILES

| | | |
|---|---|---|
| | BARCODE | CONCENTRATION |
| DYE | LEVEL | (μM) |

BIODIPY ® D-2190
(NONPOLAR) EX 493
NM/EM 503 NM

| | | |
|---|---|---|
| | 1 | 1 |
| | 2 | 0.43 |
| | 3 | 0.1 |
| | 4 | 0.043 |
| | 5 | 0.01 |
| BIODIPY ® D-2221 | | |
| (PROPIONIC ACID) | | |
| EX 460 NM/EM 570 NM | | |
| | 1 | 159 |
| | 2 | 68 |
| | 3 | 16 |
| | 4 | 6.8 |
| | 5 | 1.6 |
| BIODIPY ® D-2227 | | |
| (PROPIONIC ACID) | | |
| EX 580 NM/EM 590 NM | | |
| | 1 | 132 |
| | 2 | 57 |
| | 3 | 13 |
| | 4 | 5.7 |
| | 5 | 1.3 |
| BIODIPY ® B-3932 | | |
| (NONPOLAR) | | |
| EX 665 NM/EM 676 NM | | |
| | 1 | 100 |
| | 2 | 43 |
| | 3 | 10 |
| | 4 | 4.3 |
| | 5 | 1 |

Next, the fluorescence intensity of each dye was characterized in isolation of the others, at five different levels. Table 3 summarizes the resulting fluorescence levels detected in four different windows—FL1 (525 nm+/−10 nm), FL2 (575 nm+/−7 nm), FL3 (620 nm+/−13 nm) and FL4 (675 nm +/−15 nm). For each of the four dyes, the fluorescence intensity decreased proportionally to the decreasing dye of the bead. Moreover, each dye provided a suitably distinct fluorescence signature.

Next, the four selected dyes were mixed in varying combinations of dyes/intensity levels, as shown in Table 3. The resulting fluorescence intensities were as shown, demonstrating that the resulting beads provided discernable labeling information regarding both dye concentration and composition.

TABLE 3
FOUR DYE FLUORESCENCE CODING

| BODIPY 493N | BODIPY 560PA | BODIPY 580PA | BODIPY 665N | | | | |
|---|---|---|---|---|---|---|---|
| LEVELS | LEVELS | LEVELS | LEVELS | FL1 | FL2 | FL3 | FL4 |
| 1 | | | | 537 | 8.5 | 4.8 | 1 |
| 2 | | | | 248 4 | 3.8 | 2.2 | 1 |
| 3 | | | | 45 | 1.2 | 1.1 | 1 |
| 4 | | | | 20.4 | 1.1 | 1.1 | 1 |
| 5 | | | | 3 9 | 1 | 1 | 1 |
| | 1 | | | 43.9 | 304.2 | 427.8 | 17.2 |
| | 2 | | | 19 4 | 124 4 | 180.6 | 7 |
| | 3 | | | 3.3 | 20.7 | 33.5 | 1.4 |
| | 4 | | | 1 5 | 8.6 | 13.9 | 1.1 |
| | 5 | | | 1.3 | 2.3 | 5.3 | 1 |
| | | 1 | | 94 9 | 20 1 | 345 | 19.7 |
| | | 2 | | 37.4 | 8.2 | 140.4 | 7.8 |
| | | 3 | | 6.3 | 1.6 | 30 | 17 |
| | | 4 | | 2.5 | 1 2 | 12.8 | 1.1 |
| | | 5 | | 1.2 | 1 1 | 3 4 | 1 |
| | | | 1 | 4 | 1.6 | 11 2 | 55.8 |
| | | | 2 | 1.7 | 1 2 | 5.3 | 25.6 |
| | | | 3 | 1.1 | 1 | 2.3 | 5.3 |
| | | | 4 | 1 | 1 | 1.7 | 2.3 |
| | | | 5 | 1 | 1 | 1.4 | 1 |
| 1 | 1 | | | 505 4 | 294 3 | 446 3 | 18.8 |
| 1 | | 1 | | 529.7 | 24.8 | 334.7 | 19.2 |
| 1 | | | 1 | 450.5 | 7 8 | 14.5 | 55 4 |
| | 1 | 1 | | 117.6 | 299.7 | 796.1 | 41.6 |
| | 1 | | 1 | 41 | 234 5 | 361 4 | 74 9 |
| | | 1 | 1 | 63 | 15.7 | 260.6 | 74 7 |
| 4 | 1 | | | 60.3 | 268.8 | 443 8 | 20.4 |
| 4 | | 1 | | 87.5 | 18 | 326.4 | 20.3 |
| 4 | | | 1 | 22.2 | 2.1 | 11.4 | 58.2 |
| 1 | 4 | | | 505.8 | 12 8 | 20 1 | 1.3 |
| | 4 | 1 | | 75.4 | 23 5 | 363 2 | 22.8 |
| | 4 | | 4 | 4.8 | 5.5 | 22.3 | 60.5 |
| 1 | | 4 | | 497.5 | 8.5 | 16.2 | 1.3 |
| | 1 | 4 | | 43.9 | 266.6 | 454 | 21.5 |
| | | 4 | 1 | 5.5 | 2 2 | 19.8 | 59.6 |
| 1 | | | 4 | 489.8 | 7.8 | 5 | 2.9 |
| | 1 | | 4 | 41.4 | 261.7 | 438.5 | 22 9 |
| | | 1 | 4 | 72.7 | 17.6 | 327.8 | 22.8 |

Oligonucleotide-tagged Beads.

In some embodiments, it is possible to construct a sufficient number of unique oligonucleotide tags and to attach such tags to the randomizable supports by, e.g., linking the oligonucleotide to a biotin linker and adhering that linker to a streptavidin-coated bead such as those described above. The oligonucleotide tags bear unique DNA sequences, each of which can be correlated to a given ligand.

Such DNA tags can be built in one of several ways. For example, using techniques well known to the art, a multichannel oligonucleotide synthesizer can generate a set of DNA molecules with unique sequences of any given length. Once individual oligonucleotide tags characterized and isolated into homogeneous tag pools, the tags can be adhered to the randomizable supports in a variety of ways. For example, if the randomizable supports have a streptavidin coating, then a biotin adhesion moiety is joined to each oligonucleotide tag at the 5′ end by standard synthesis techniques. If the randomizable support is coated with other adhesion moieties, the complementary adhesion moiety can be chemically coupled to a 5′ amino-modified oligonucleotide tag.

The oligonucleotide tags may be read either by sequencing, by evaluating sequence length, or by hybridization. For sequencing information, the oligonucleotide tags resident on each bead are subjected to PCR, and then run on a sequencing gel. Alternatively, the oligonucleotide tags may be identified via exposing the tags to known hybridization probes.

Mass Spectrometry Tags

Another suitable method for encoding identities of beads involves use of mass tags—i.e., labels that can be detected by mass spectrometry. Such mass tags are known in the art and must be coupled to the beads in different amounts so as to generate a mass tag bar code. This code can be read by subjecting the beads to mass spectrometry pursuant to methods familiar to those of the art, or by use of gas chromatography.

Radio-frequency Tags.

As yet another alternative for encoding identity information on beads, the beads may be engineered to emit unique, identifying radio signals of various predetermined frequencies. Such beads may contain, e.g., miniaturized transmitter/receiver circuitry, rectifier, control logic and antenna. Each set of beads thus may contain a unique label laser-etched on the internal chip within the bead. Emissions from the radio-frequency tags are detected by a corresponding radio-frequency detector.

Beads with Mixed Tags.

In some embodiments, the number of different ligand populations to be uniquely tagged will be quite large—on the order of 1×10⁶ or more. Although a corresponding number of unique fluorescent bar code tags, mass spec tags or DNA oligonucleotide tags could be formulated as described above, in some instances it may be desirable to make tags that are some combination of fluorescent, mass spec and/or oligonucleotide information. For example, oligonucleotide tags or mass spec tags may be incorporated so as to reduce the number of fluorescent dyes used. Such techniques may advantageously reduce or avoid any instances of fluorescent quenching or fluorescence resonance energy transfer (FRET), and/or may expand the number of bar codes that can be used.

Bead-polypeptide Interactions

To test the bead:bead interactions of the invention, several proteins were inserted into pET-biotrx-BirA and overexpressed in BL21 (DE3) RIL cells: murine p53, SV40 large T-antigen, HPV16 E7 and the “Rb pocket” of the Retinoblastoma gene. The E7 and p53 polypeptides were bound to the beads via the associated biotinylation signal, and were detected on the beads with antibidies specific to E7 and p53, respectively.

### EXAMPLE 3

### High Throughput Screening of a Comprehensive Human Protein Library for Protein-Protein Interactions

The goal of the process is to examine in a quantitative or semi-quantitative fashion all possible pairwise interactions between human protein domains. This involves a test of “n×n” interactions, if “n” is the number of human protein domains. Values for “n” likely fall between 100,000 and 1,000,000. For an interaction screen of this scope, automation of at least some of the following procedures is desirable.

To summarize, one embodiment of the process involves a series of steps: (1) generation of a library of expressed human sequences in an E. coli expression vector such that the human DNA is expressed as a fusion with a suitable adhesion moiety; in addition, part of the fusion protein may serve as a recognition sequence tag for attaching labels (e.g., fluorescent antibody labels) so that the protein can be detected; (2) enrichment of the library for clones that contain constructs that are in-frame and expressed at reasonable levels; (3) arraying of the enriched library clones in microtiter plates; (4) growth and induction of the individual library clones to produce fusion proteins inside E. coli; (5) preparation of E. coli lysates to release the expressed fusion proteins from cells; (6) generation of a primary set of beads barcoded with suitable combinations of fluorescent dyes to act as randomizable supports; (7) apportioning of beads to individual wells of microtiter trays to permit adhesion of lysate fusion proteins to the randomizable supports (also referred to herein as “primary beads”); (8) apportionment of secondary magnetic beads (as location-determinable supports) to microtiter wells to allow adhesion of lysate proteins as in 7; (9) mixing of primary and secondary beads to permit aggregation of beads with interacting proteins on their surfaces; (10) magnetic capture of secondary beads and attached primary beads to enrich for primary beads with proteins that interact with protein on the surface of secondary beads; (11) mixing of enriched primary beads with soluble fusion protein in microtiter wells to allow interaction of soluble protein with proteins on the surface of primary beads, as well as detachment of secondary beads; (13) magnetic capture and disposal of secondary beads; (14) collection of primary beads and crosslinking of bound protein using, e.g., paraformaldehyde; (15) exposure to labeling agent (e.g., fluorescent antibody) to enable detection of bound secondary proteins; and (16) detection of labeling agent and barcode reading to determine identity of primary protein (on bead surface) and amount of secondary protein attached via interaction with primary protein. Other embodiments may add to, alter or delete some of the above steps, in ways that will be apparent to one of ordinary skill in the art.

Steps 1 and 2—generation and enrichment of the polypeptide library to be cross-screened in order to generate a protein interaction may—is described in detail in Example 1, above.

Step 3 involves plating out and growing up single-cell clones that produce only one of the library polypeptides at a given unique array location. To accomplish this, a commercial robot may be used (e.g., Genetix Ltd. “Q-bot™; TM Analytic, PBA Flexys™; BioRobotics Ltd, BioPick™; or Linear Drives Ltd., Mantis™; any of which with multiple pin tool picking head) to select out a single colony and transfer the cells to a corresponding unique array location in e.g., a 384 well microtiter plate (40 μl volume). Each clone in the array is grown in, e.g., Luria broth or minimal media until early- to mid-log phase, and then expression of the human protein domain construct is induced by adding IPTG (step 4). After a suitable period of time to allow polypeptide expression, the cells are then lysed (step 5) by the method described in detail in Example 1. Thus, each unique location in the chosen array format (384 well plate or other) will contain a lysate bearing one particular human protein domain, amongst the milieu of native E. coli proteins.

Alternatively, for ease of generating and processing the lysate from the single cell clone, a single colony may be picked and transferred to a correspondingly unique intermediate container of larger volume for growing up the clone. Once the clone is finished culturing, a sample is taken from the intermediate container and is concentrated and lysed as described in detail in Example 1. An aliquot of the lysate is then transferred to a unique array location in a 384 well microtiter plate (40 μl volume).

Step 6 involves the generation of the primary set of beads with fluorescent barcodes. These beads are the randomizable supports that will allow presentation of an aliquot bearing a fully integrated collection of lysate protein domains to each such domain independently, to map all possible interactions amongst those protein domains. Example 2 describes preparation of these uniquely tagged fluorescent beads in detail.

Once each primary set of beads with a corresponding unique fluorescent tag is generated, the bead sets are suspended in buffer. A sampling from each tagged bead set is then dispersed into a corresponding array location, so that the tagged primary beads adhere to the protein domains therein (step 7). This may be accomplished by e.g., automated aspiration of the beads into the wells (e.g., TecanAG Genesis™; Matrix Technologies Corp. PlateMate™; Carl Creative Systems, Inc. PlateTrak™) or hopper release of beads into wells. Conversely, an aliquot of the lysate may be aspirated or released from a hopper into a corresponding microtiter well that already contains these primary fluorescent beads. In either event, the beads and protein domains are brought into contact and allowed to adhere via the adhesion moiety fused to the protein domain. The identity of the adhered protein thereafter can be determined via the corresponding, unique fluorescent bar code tag on the bead.

Once each of the unique array locations (i.e., polypeptides or other substrates) has been exposed to a corresponding set of beads bearing a unique tag, all beads are collected and mixed to form a fully integrated set of protein-bearing beads. This random mixing is accomplished by multiple, automated aspiration and release cycles, by plate agitation with a robotic shaker, or by mechanical stirring.

Next, the secondary set of magnetic beads are prepared in situ in each of the unique locations in the library array (step 8). This is accomplished by adding an aliquot of beads to each library as in step 7. Alternatively, a robotic hand with magnetized fingers may be used to capture the magnetic beads and then release. the beads in each of the corresponding array locations on the, e.g., 384 well plate, by dipping the fingers into the lysate and demagnetizing the fingers.

Aliquots taken from the fully integrated set of primary beads are then collected and dispensed into each unique array location, each of which contains a location-determinable set of secondary beads with adhered protein domains (step 9). The number of primary beads (i.e. randomizable substrates) should be sufficient to reduce probability of not having a particular polypeptide/bead to a small value—e.g., less than 1:100 probability. This may be accomplished by aspirating and dispensing, as above. This step allows complexes to form between the protein domains adhered to the primary and secondary beads at each array location, and hence forming bead-bead aggregates.

Complexes of adhered beads are then retrieved magnetically (step 10) with, e.g., a neodymium-iron-boron magnet (Master Magnetics Inc.). The magnetic aggregates using relatively large magnetic beads (i.e. larger than about 50 nm diameter) are magnetically attracted to the sides of the microtiter wells, either on one side or around the entire perimeter of the wells. Remaining beads are washed away. As yet another alternative, a ferromagnetic pin is placed in the center of the well, with magnets located on the outside of the well. Geometry of the pin and magnet is selected so that the induced magnetic field on the pin will attract the beads, and beads that do not react are removed.

Quantification of polypeptide-ligand complexes may be facilitated by replacement of bead-bound protein domain with a soluble, unbound form of the domain (step 11). This is accomplished by introducing the enriched bead complexes derived from step 10 into a soluble protein domain lysate that matches the protein domain on the secondary bead (i.e., the location-determinable domain). Alternatively, the beads may be exposed to the products of a separate library that contains polypeptide inserts that correspond to each polypeptide moiety that is adhered to the bead, but which has a unique labeling domain or epitope. This is readily accomplished by placing the complexes that correspond to, e.g., an array location designated “1” in a first a set of primary 384-well microtiter trays (step 3) into a corresponding location, e.g., designated “1′”, of a duplicate microtiter tray that was prepared in parallel in step 3. Since array location 1 and 1′ contain the same lysate, the free lysate in 1 will competitively displace the bead-bound lysate of the complex. As a result, the primary bead will now bear two layers of protein domains, adhered to one another via protein-protein interactions.

Once the protein-protein interactions are established, the primary beads are collected in a manner that segregates the beads in groups that correspond to each separate array location from which the protein bound to the secondary bead originated and the bound proteins crosslinked with, e.g., paraformaldehyde (step 14) to stabilize the complexes by preventing dissociation.

These stabilized protein-protein pairs are then exposed to a fluorescent antibody (step 15). As one non-limiting example, one may detect a bound secondary protein by using a fluorescently-labeled antibody directed against one of the fusion protein epitopes (used as a recognition domain and shared among all library constructs), e.g., a FLAG or biotin epitope. The antibody is incubated with the crosslinked beads, such that it binds to exposed or unique epitopes on the secondary protein; i.e., the labeling agent must recognize an epitope that is either absent from the primary fusion polypeptide, thus necessitating construction and array of a separate library for the secondary polypeptide, or an epitope that is inaccessible on the primary polypeptide). Alternatively, fluorescently labeled avidin may be used. These beads are washed in binding buffer and then analyzed as described below. The fluorescence intensity of the antibody fluorochrome serves as a surrogate for the amount of bound secondary protein.

Finally, in step 16, the beads bearing these segregated, labeled protein pairs are then examined by a detecting device to quantify conjugates that have the antibody or biotin label. In one preferred embodiment, the fluorescence information (both wavelength and intensity signatures) are simultaneously read and used to identify the protein domain adhered to that bead. Alternatively or in conjunction, the beads are decoded using familiar techniques such as sequencing or hybridization of oligonucleotide tags, or mass spectrometry to identify mass tags.

This sorting and/or detection step can be accomplished via one of a number of instruments. Two general categories of instrument have particular utility: a flow cytometry instrument such as a FACS machine or flow analyzer; CCD detector or photomultiplier tube scanner. Each device must have certain capabilities. It must permit rapid analysis of beads using, in the case of FACS, multiple lasers for excitation (e.g., three lasers), and detection of fluorescent emissions at multiple wavelengths (e.g., 3-10 wavelengths). Such capabilities presently exist in the Cytomation flow sorter. The three lasers excite cells or beads in liquid droplets sequentially as the droplets fall in a stream. A series of filters and photo-multiplier tubes (PMTs) then collect emitted light at different preselected wavelengths. These data are stored and can be accessed for analysis later off-line from files.

The bead barcode reveals the identity of the primary protein by correlating that protein back to a unique library array location—i.e. the microtiter well that contained the one particular lysate that was exposed to that barcoded primary bead. This barcode is read in the same step as the antibody quantitation is performed. However, to decode a large number of bar codes, multiple measurements on each bead are required. For example, it may be necessary to measure fluorescence emissions of ten dyes at ten wavelengths with specific excitation lasers. These ten measurements provide sufficient information to unambiguously identify each bead according to its specific barcode.

The process by which this computation is performed involves two basic steps: (1) parameters are fit to known barcode data; (2) the fitted parameters are used in a deconvolution calculation to determine the bar codes of unknown beads. Total fluorescence of a barcoded bead at a particular wavelength (and at a particular excitation wavelength) can be calculated according to a formula:

F=1₁f₁+1₂f₂+. . . +1ₙfₙ

where 1₁ is the quantity or level of the first dye and f₁ is the normalized fluorescence contribution of the first dye under particular conditions of excitation and emission (i.e., wavelengths). By generating many beads with defined dye ratios (i.e., bar codes) and measuring their fluorescence (F) at specific wavelengths, it is possible to fit the fₙ parameters and create at specific wavelengths a set of equations that relate total fluorescence to the individual fluorescences of the different dyes. After this is completed, it is possible to calculate the 1ₙ's of an unknown bead, thereby determining its barcode and identity. It is necessary to have at least as many independent measurements of F (i.e., at different excitation/emission wavelengths) as there are unknown “1” values in the bar code.

The fluorescent barcode is used to determine the bead identity, an identity that is linked to the well from which it was originally derived; that is, a barcode matches a well which contained the lysate fusion protein that comprises layer one on the bead. Thus, the nature of the first layer of protein that is adhered to the support can be determined by DNA sequence analysis of the cloned insert in each well. This sequence analysis can be accomplished simply by PCR amplification of insert sequences from each microtiter well using primers on the vector which flank the insert. Standard automated sequence analysis followed by database searches reveals details about each cloned insert. Current sequencing throughputs permit sequencing of one million inserts in a period of weeks to months.

As described above, the fluorescence of a labeling agent, e.g., an antibody against a FLAG epitope serves to quantify the amount of secondary protein attached via protein-protein interactions to a bead. If the concentration of protein in the lysate is measured or estimated, and the saturating amount of protein on the bead is known (i.e., how much secondary protein could be maximally bound if all primary protein binding sites were occupied), it is possible to determine the approximate binding constant of the protein-protein interaction from the equation:

K_{d}=[xy]/[x][y]

where the ratio [xy]/[x] is simply the ratio of measured bound secondary protein over the saturating (maximal) bound amount, and [y] is concentration of soluble fusion protein in the lysate.

While the present invention has been described in terms of specific methods and compositions, it is understood that variations and modifications will occur to those skilled in the art in consideration of the present invention. Accordingly. it is intended in the appended claims to cover all such equivalent variations which come within the scope of the invention as claimed, in light of,those variations and modifications.

1. A method for identifying interacting substrate-ligand pairs, comprising the steps of:
(a) adhering a plurality of ligands to a corresponding plurality of randomizable supports bearing a unique fluorescent dye identifier;
(b) contacting said ligands with a substrate derived from a unique location so as to form at least one substrate/ligand complex;
(c) identifying any complex-forming ligand by its corresponding unique fluorescent dye identifier; and
(d) identifying any complex-forming substrate by determining its corresponding unique location.

2. The method of claim 1, wherein said substrate is an individual polypeptide.

3. The method of claim 1, wherein said substrate is a library polypeptide.

4. The method of claim 3, wherein said library polypeptide is a native polypeptide.

5. The method of claim 3, wherein said library polypeptide is a member of a large library.

6. The method of claim 3, wherein said library polypeptide is a member of a very large library.

7. The method of claim 3, wherein the identity of said library polypeptide is not known prior to step (a).

8. The method of claim 1, wherein said ligands are polypeptides.

9. The method of claim 8, wherein said ligands are library polypeptides.

10. The method of claim 9, wherein said library polypeptides are native polypeptides.

11. The method of claim 9, wherein said library polypeptides are members of a large library.

12. The method of claim 9, wherein said library polypeptides are members of a very large library.

13. The method of claim 8, wherein the identities of said polypeptides are not known prior to step (a).

14. The method of claim 7, wherein each said substrate derived from a unique location is adhered to a corresponding location determinable support.

15. The method of claim 1 wherein said randomizable support is magnetized and said complexes are segregated by being magnetically culled.

16. The method of claim 14, wherein said location determinable support is magnetized and said complexes are segregated by being magnetically culled.

17. The method of claim 15, wherein said randomizable supports are beads.

18. The method of claim 1, wherein said unique fluorescent dye identifier is comprises a plurality of fluorescent dye species.

19. The method of claim 18, wherein said plurality of fluorescent dye species includes at least one species of fluorescent nanoparticle.

20. The method of claim 18, wherein said plurality of fluorescent dye species includes at least one species of organic dye.

21. The method of claim 20, wherein said organic dye species is selected from the group consisting of the organic dyes listed in Table 1.

22. The method of claim 1, wherein said ligands are non-proteinaceous organic molecules.

23. The method of claim 1, wherein said step of identifying comprises the step of detecting each said substrate/ligand complex with a fluorescent label.

24. The method of claim 22, further comprising the step of detecting said substrate/ligand complex with a CCD camera..

25. A human protein interaction map produced by the method of claim 1.
